(19) United States
(12) Patent Application Publication
(10) Pub. No.: US 2026/0242761 A1
(43) Pub. Date: Aug. 20, 2026

(54) FUSION PROTEINS AND APPLICATIONS THEREOF

(71) Applicant: GUANGZHOU REFORGENE MEDICINE CO., LTD., Guangzhou (CN)

(72) Inventors: Junbin LIANG, Guangzhou (CN); Liancheng HUANG, Guangzhou (CN); Hui XU, Guangzhou (CN); Jinxiu CAI, Guangzhou (CN); Zhengxia YANG, Guangzhou (CN)

(73) Assignee: GUANGZHOU REFORGENE MEDICINE CO., LTD., Guangzhou (CN)

(21) Appl. No.: 19/568,730

(22) Filed: Mar. 16, 2026

Related U.S. Application Data
(63) Continuation of application No. PCT/CN2024/120186, filed on Sep. 20, 2024.

(30) Foreign Application Priority Data
Sep. 20, 2023 (CN) 202311212915.4

(51) Int. Cl. C12N 9/22; A61K 38/00; C12N 15/11

(52) U.S. Cl. CPC C12N 9/226; C12N 15/111; A61K 38/00; C12N 2310/20; C12Y 301/21004

(57) ABSTRACT

The present disclosure discloses a fusion protein and use thereof. The fusion protein is obtained by replacing amino acid residues 943-952 of a nuclease-inactivated mutant of a Cas9 protein having the amino acid sequence as set forth in SEQ ID NO: 1 with a functional domain. The fusion protein of the present disclosure is used for gene editing, and can achieve highly specific cleavage, high editing efficiency, and low off-target risk.

### CROSS-REFERENCE TO RELATED APPLICATIONS

This application is a continuation of International Application No. PCT/CN2024/120186, filed on Sep. 20, 2024, which claims priority to Chinese patent application No. 202311212915.4 filed on Sep. 20, 2023. The entirety of the above Chinese patent application is incorporated herein by reference.

### SEQUENCE LISTING

The instant application contains a sequence listing which has been submitted electronically in XML format and is hereby incorporated by reference in its entirety. The XML copy, created on Mar. 13, 2026, is named “SEQUENCE_LISTING-20954-0007US00” and is 107,455 bytes in size.

### TECHNICAL FIELD

The present disclosure belongs to the field of biotechnology, and in particular relates to a fusion protein and use thereof.

### BACKGROUND

CRISPR-Cas9 is an adaptive immune defense formed in bacteria and archaea during long-term evolution, and can be used to combat invading viruses and exogenous DNA. SpCas9 in the CRISPR/Cas9 system derived from Streptococcus pyogenes has been widely used in genetic engineering because it is simple to operate and efficient. The CRISPR-Cas9 system consists of Cas9, crRNA, and tracrRNA, wherein the Cas9 protein is a DNA endonuclease that can be expressed by many bacteria, and the Cas9 endonuclease must cleave DNA under the guidance of a gRNA molecule (a complex of crRNA and tracrRNA); Cas9, crRNA, and tracrRNA first bind to form a complex, recognize and bind to the complementary target sequence of the crRNA, and then the active site of the HNH nuclease domain of the Cas9 protein cleaves the DNA strand complementary to the crRNA, while the active site of the RuvC nuclease domain cleaves the non-complementary strand, thereby causing double-strand breaks in DNA and achieving an editing effect on specific DNA through NHEJ or HDR.

### SUMMARY

The present disclosure provides a fusion protein and use thereof. The fusion protein of the present disclosure is used for gene editing, and can achieve highly specific cleavage, high editing efficiency, and/or low off-target risk.

In order to improve DNA cleavage specificity, we generated a fusion of catalytically inactive Cas9 and the FokI nuclease, and designed, screened, and tested the position of FokI in the fusion protein, ultimately obtaining a novel fusion protein which, in some examples, exhibits editing activity superior to currently known Cas9 proteins having terminally fused FokI.

In some cases, the indel efficiency of the dCas9-FokI of the present disclosure in combination with a pair of gRNAs is even higher than that of wild-type SpCas9.

Those skilled in the art will also appreciate that the dCas9-FokI of the present disclosure, in combination with a pair of gRNAs, generates two single-strand breaks on complementary DNA strands, thereby forming a double-strand break. As compared with wild-type SpCas9, which directly introduces a double-strand break, the dCas9-FokI of the present disclosure is expected to reduce off-target activity. See, for example, Maryam Saifaldeen et al., CRISPR FokI Dead Cas9 System: Principles and Applications in Genome Engineering, Cells, 2020, 9, 2518.

In a first aspect, the present disclosure provides a fusion protein, wherein the fusion protein is obtained by replacing consecutive amino acid residues in a random coil structure of dCas9 (also referred to as dCas9 protein) with a functional domain, and the dCas9 is a Cas9 protein having no nuclease activity;
optionally, the functional domain is selected from a deaminase domain (including but not limited to a cytidine deaminase domain and an adenine deaminase domain), a UGI domain, a UDG domain, a methylation domain, a demethylation domain, a subcellular localization signal (including but not limited to a nuclear localization signal, a nuclear export signal, a mitochondrial localization signal, and a chloroplast localization signal), a transcriptional activation domain, a transcriptional repression domain, a nuclease domain (including but not limited to a FokI domain), a histone deacetylation domain, and a DNA ligation domain; further optionally, the functional domain is a nuclease domain; still further optionally, the functional domain is a FokI domain;
optionally, the random coil structure is located in the REC1-A and/or REC1-B domain of dCas9;
optionally, the random coil structure is located in the RuvC, loop L2, and/or HNH domain of dCas9; or the random coil structure is located in the RuvC domain of dCas9; or the random coil structure is located in the RuvC-III domain of dCas9.

In a second aspect, the present disclosure provides a fusion protein, wherein the fusion protein comprises dCas9 portion and a functional domain, and the dCas9 portion is a remaining part of a dCas9 protein in which one or more consecutive amino acid residues located in a random coil region of the dCas9 protein are replaced by the functional domain; the dCas9 is a Cas9 protein having no nuclease activity;
optionally, the functional domain is selected from a deaminase domain (including but not limited to a cytidine deaminase domain and an adenine deaminase domain), a UGI domain, a UDG domain, a methylation domain, a demethylation domain, a subcellular localization signal (including but not limited to a nuclear localization signal, a nuclear export signal, a mitochondrial localization signal, and a chloroplast localization signal), a transcriptional activation domain, a transcriptional repression domain, a nuclease domain (including but not limited to a FokI domain), a histone deacetylation domain, and a DNA ligation domain; further optionally, the functional domain is a nuclease domain; still further optionally, the functional domain is a FokI domain;
optionally, the random coil structure is located in the REC1-A and/or REC1-B domain of dCas9;
optionally, the random coil structure is located in the RuvC, loop L2, and/or HNH domain of dCas9; or the random coil structure is located in the RuvC domain of dCas9; or the random coil structure is located in the RuvC-III domain of dCas9.

In some embodiments of the present disclosure, the fusion protein is capable of specifically binding to a target nucleic acid under the guidance of a guide polynucleotide.

In some embodiments of the present disclosure, the fusion protein is capable of binding to a target nucleic acid in a sequence-specific manner under the guidance of a guide polynucleotide, wherein the guide polynucleotide comprises a guide sequence, and the guide sequence hybridizes to the target nucleic acid.

In some embodiments of the present disclosure, the fusion protein is capable of forming a complex with a guide polynucleotide, wherein the guide polynucleotide comprises a guide sequence, and the guide sequence is engineered to direct the complex to bind to a target nucleic acid in a sequence-specific manner.

In some specific embodiments of the present disclosure, the functional domain is a FokI domain, and the fusion protein is capable of recognizing a double-stranded target nucleic acid and nicking the target nucleic acid under the guidance of a guide polynucleotide; or the fusion protein is capable of forming a complex with a guide polynucleotide, wherein the guide polynucleotide comprises a guide sequence, and the guide sequence is engineered to direct the complex to bind to a double-stranded target nucleic acid in a sequence-specific manner and nick the target nucleic acid.

In some preferred embodiments of the present disclosure, the guide polynucleotide further comprises a scaffold sequence; further, the scaffold sequence interacts with the dCas9 portion of the fusion protein, or the scaffold sequence enables the guide polynucleotide to form a complex with the fusion protein.

In some embodiments of the present disclosure, the Cas9 protein is selected from SpCas9, SaCas9, NmeCas9, StCas9, and CjCas9.

In some preferred embodiments of the present disclosure, the Cas9 protein is SpCas9 (Streptococcus pyogenes Cas9) or SaCas9 (Staphylococcus aureus Cas9).

In some specific embodiments of the present disclosure, the amino acid sequence of the SpCas9 is as set forth in SEQ ID NO: 1.

In some embodiments of the present disclosure, the amino acid sequence of the Cas9 protein has ≥80%, ≥85%, ≥90%, ≥95%, ≥96%, ≥97%, ≥98%, ≥99%, or ≥99.5% sequence identity to the amino acid sequence as set forth in SEQ ID NO: 1.

In some specific embodiments of the present disclosure, the amino acid sequence of the dCas9 is as set forth in SEQ ID NO: 2, or has ≥80%, ≥85%, ≥90%, ≥95%, ≥96%, ≥97%, ≥98%, ≥99%, or ≥99.5% sequence identity to the amino acid sequence as set forth in SEQ ID NO: 2.

In some embodiments of the present disclosure, the dCas9 comprises a mutation, for example a point mutation, in the RuvC domain and/or HNH domain of the Cas9 protein, such that the Cas9 protein has no nuclease activity.

In some preferred embodiments of the present disclosure, the dCas9 is a Cas9 in which the positions corresponding to D10 and N863 of SpCas9 are mutated to A; or the dCas9 is a Cas9 in which the positions corresponding to D10 and H840 of SpCas9 are mutated to A. The corresponding positions can be determined by sequence alignment of the Cas9 with SpCas9.

In some preferred embodiments of the present disclosure, the dCas9 is SpCas9 having D10A and N863A mutations, or SpCas9 having D10A and H840A mutations.

In some preferred embodiments of the present disclosure, the dCas9 is SaCas9 having D10A and H557A mutations.

In some embodiments of the present disclosure, the functional domain is fused directly or via a linker to dCas9 portion. In some embodiments of the present disclosure, the functional domain is fused directly to dCas9 portion (that is, the N-terminal residue of the functional domain is directly covalently linked by a peptide bond to the N-terminal residue of the split dCas9 portion, and the C-terminal residue of the functional domain is directly covalently linked by a peptide bond to the C-terminal residue of the split dCas9), or the functional domain is fused to dCas9 through an amino acid sequence. In some embodiments of the present disclosure, the functional domain is fused directly to dCas9 portion, or the functional domain is fused to dCas9 portion through a linker sequence having a length of 1-50, 1-40, 1-30, 1-20, 5-16, 1-10, 10-20, 3-8, or 15-20 amino acid residues.

In some specific embodiments of the present disclosure, the linker is an amino acid residue sequence GGSGS (SEQ ID NO: 50) or SGSETPGTSESATPES (SEQ ID NO: 51).

In some embodiments of the present disclosure, the fusion protein is obtained by replacing 1-20 consecutive amino acid residues of a random coil structure of dCas9 with the functional domain.

In some embodiments of the present disclosure, the fusion protein is obtained by replacing 1, 2, 3, 4, 5, 6, 7, 8, 9, 10, 11, 12, 13, 14, 15, 16, 17, 18, 19, or 20 consecutive amino acid residues of a random coil structure of dCas9 with the functional domain.

In some embodiments of the present disclosure, the fusion protein is obtained by replacing 1-15, 3-11, 3-10, or 9-11 consecutive amino acid residues of a random coil structure of dCas9 with the functional domain.

In some embodiments of the present disclosure, the replaced random coil structure has a length of 1-15 amino acid residues.

In some specific embodiments of the present disclosure, the replaced random coil structure has a length of 3-11 amino acid residues.

In some other specific embodiments of the present disclosure, the replaced random coil structure has a length of 3-10 amino acid residues.

In some other specific embodiments of the present disclosure, the replaced random coil structure has a length of 9-11 amino acid residues.

In some other specific embodiments of the present disclosure, the replaced random coil structure has a length of 3 amino acid residues.

In some embodiments of the present disclosure, the random coil structure corresponds to positions 37-41, 107-113, 147-149, 169-172, 308-314, 792-799, 864-871, 906-908, 943-952, and/or 1015-1017 of the sequence shown in SEQ ID NO: 2.

In some preferred embodiments of the present disclosure, when the amino acid sequence of the dCas9 is as set forth in SEQ ID NO: 2, the replacement occurs at positions 37-41, 107-113, 147-149, 169-172, 308-314, 792-799, 864-871, 906-908, 943-952, or 1015-1017.

In the present disclosure, the position of the random coil structure can be determined by sequence alignment with the sequence as set forth in SEQ ID NO: 2.

In some embodiments of the present disclosure, after replacement of the sequence to be replaced in the dCas9, the functional domain is fused directly or via a linker to dCas9 portion.

In some specific embodiments of the present disclosure, the sequence to be replaced has a length of 1-15 amino acid residues.

In some other specific embodiments of the present disclosure, the sequence to be replaced has a length of 3-11 amino acid residues.

In some other specific embodiments of the present disclosure, the sequence to be replaced has a length of 3-10 amino acid residues.

In some other specific embodiments of the present disclosure, the sequence to be replaced has a length of 9-11 amino acid residues.

In some other specific embodiments of the present disclosure, the sequence to be replaced has a length of 3 amino acid residues.

In some specific embodiments of the present disclosure:
when the sequence to be replaced is located at positions 37-41 of the amino acid sequence as set forth in SEQ ID NO: 2, the sequence to be replaced has a length of 5 amino acid residues;
when the sequence to be replaced is located at positions 107-113 of the amino acid sequence as set forth in SEQ ID NO: 2, the sequence to be replaced has a length of 7 amino acid residues;
when the sequence to be replaced is located at positions 147-149 of the amino acid sequence as set forth in SEQ ID NO: 2, the sequence to be replaced has a length of 3 amino acid residues;
when the sequence to be replaced is located at positions 169-172 of the amino acid sequence as set forth in SEQ ID NO: 2, the sequence to be replaced has a length of 4 amino acid residues;
when the sequence to be replaced is located at positions 308-314 of the amino acid sequence as set forth in SEQ ID NO: 2, the sequence to be replaced has a length of 7 amino acid residues;
when the sequence to be replaced is located at positions 792-799 of the amino acid sequence as set forth in SEQ ID NO: 2, the sequence to be replaced has a length of 8 amino acid residues;
when the sequence to be replaced is located at positions 864-871 of the amino acid sequence as set forth in SEQ ID NO: 2, the sequence to be replaced has a length of 8 amino acid residues;
when the sequence to be replaced is located at positions 906-908 of the amino acid sequence as set forth in SEQ ID NO: 2, the sequence to be replaced has a length of 3 amino acid residues;
when the sequence to be replaced is located at positions 943-952 of the amino acid sequence as set forth in SEQ ID NO: 2, the sequence to be replaced has a length of 10 amino acid residues;
when the sequence to be replaced is located at positions 1015-1017 of the amino acid sequence as set forth in SEQ ID NO: 2, the sequence to be replaced has a length of 3 amino acid residues.

In some embodiments of the present disclosure, the FokI domain comprises the DNA-binding domain and cleavage domain of the FokI restriction endonuclease.

In some embodiments of the present disclosure, the FokI domain comprises, or is, the DNA cleavage domain of the FokI restriction endonuclease.

In the present disclosure, the FokI domain may exist in monomeric or dimeric form. For example, the FokI domain may exist in monomeric form in solution.

In some embodiments of the present disclosure, the FokI domain has nuclease activity.

In one example of the present disclosure, the first fusion protein can bind, together with a first guide polynucleotide, to one single strand of a double-stranded target nucleic acid, and the second fusion protein can bind, together with a second guide polynucleotide, to the other single strand of the target nucleic acid;

the FokI domains of the first fusion protein and the second fusion protein are separated by a spacer sequence on the double-stranded target nucleic acid and can form a dimer, the spacer sequence having a length of 1-100 bp, and the dimer can cleave the double-stranded target nucleic acid when the first fusion protein, the first guide polynucleotide, the second fusion protein, and the second guide polynucleotide bind to the target nucleic acid.

In one example of the present disclosure, the FokI domains may dimerize after dCas9 binds to double-stranded target DNA, thereby cleaving the double-stranded target DNA through nuclease activity thereof. When the FokI domains dimerize, two FokI domains are separated by a spacer sequence on the double-stranded target DNA so as to cleave the double-stranded target DNA. The spacer sequence herein does not refer to the spacer sequence of a CRISPR array in a CRISPR system.

In the present disclosure, the spacer sequence may have a length of 1-100 bp, 1-90 bp, 1-80 bp, 1-70 bp, 1-60 bp, 1-50 bp, 1-40 bp, 1-30 bp, 1-20 bp, 1-10 bp, 1-5 bp, 2-100 bp, 2-90 bp, 2-80 bp, 2-70 bp, 2-60 bp, 2-50 bp, 2-40 bp, 2-30 bp, 2-20 bp, 2-10 bp, 2-5 bp, 3-100 bp, 3-90 bp, 3-80 bp, 3-70 bp, 3-60 bp, 3-50 bp, 3-40 bp, 3-30 bp, 3-20 bp, 3-10 bp, 3-5 bp, 4-100 bp, 4-90 bp, 4-80 bp, 4-70 bp, 4-60 bp, 4-50 bp, 4-40 bp, 4-30 bp, 4-20 bp, 4-10 bp, 4-5 bp, 5-100 bp, 5-90 bp, 5-80 bp, 5-70 bp, 5-60 bp, 5-50 bp, 5-40 bp, 5-30 bp, 5-20 bp, 5-10 bp, 10-100 bp, 10-90 bp, 10-80 bp, 10-70 bp, 10-60 bp, 10-50 bp, 10-40 bp, 10-30 bp, 10-20 bp, 15-100 bp, 15-90 bp, 15-80 bp, 15-70 bp, 15-60 bp, 15-50 bp, 15-40 bp, 15-30 bp, 15-20 bp, 20-100 bp, 20-90 bp, 20-80 bp, 20-70 bp, 20-60 bp, 20-50 bp, 20-40 bp, 20-30 bp, or 20-25 bp. In some embodiments of the present disclosure, the spacer sequence has a length of 4-30 bp. In some embodiments of the present disclosure, the spacer sequence has a length of 6-20 bp. In some embodiments of the present disclosure, the spacer sequence has a length of 1 bp, 2 bp, 3 bp, 4 bp, 5 bp, 6 bp, 7 bp, 8 bp, 9 bp, 10 bp, 11 bp, 12 bp, 13 bp, 14 bp, 15 bp, 16 bp, 17 bp, 18 bp, 19 bp, 20 bp, 21 bp, 22 bp, 23 bp, 24 bp, 25 bp, 26 bp, 27 bp, 28 bp, 29 bp, 30 bp, 31 bp, 32 bp, 33 bp, 34 bp, 35 bp, 36 bp, 37 bp, 38 bp, 39 bp, or 40 bp. For example, the spacer sequence has a length of 4 bp, 5 bp, 6 bp, 7 bp, 8 bp, 9 bp, 10 bp, 11 bp, 12 bp, 13 bp, 14 bp, 15 bp, 16 bp, 17 bp, 18 bp, 19 bp, 20 bp, 21 bp, 22 bp, 23 bp, 24 bp, 25 bp, 26 bp, 27 bp, 28 bp, 29 bp, or 30 bp.

In some embodiments of the present disclosure, the amino acid sequence of the FokI domain has ≥50%, ≥60%, ≥70%, ≥80%, ≥90%, ≥95%, ≥98%, or ≥99% sequence identity to amino acid residues 6-201 of the sequence as set forth in SEQ ID NO: 4.

In some embodiments of the present disclosure, the FokI domain comprises amino acid residues 6-201 of the sequence as set forth in SEQ ID NO: 4.

In some embodiments of the present disclosure, the FokI domain consists of amino acid residues 6-201 of the sequence as set forth in SEQ ID NO: 4.

In some embodiments of the present disclosure, the amino acid sequence of the FokI domain is as set forth in SEQ ID NO: 52, or has ≥50%, ≥60%, ≥70%, ≥80%, ≥90%, ≥95%, ≥98%, or ≥99% sequence identity to the amino acid sequence as set forth in SEQ ID NO: 52.

In some embodiments of the present disclosure, the guide polynucleotide is a single-molecule guide polynucleotide or a dual-molecule guide polynucleotide. By way of non-limiting example, the guide polynucleotide is a single-molecule guide polynucleotide (sgRNA) formed by chimerizing crRNA and tracrRNA in a CRISPR-Cas9 system. By way of another example, the guide polynucleotide is a set composed of two separate molecules, crRNA and tracrRNA, in the CRISPR-Cas9 system, namely the dual-molecule form.

In some embodiments of the present disclosure, the fusion protein further comprises any one or more of the following: a subcellular localization signal, a DNA-binding domain, a transcriptional activation domain, a transcriptional repression domain, a nuclease domain, a deaminase domain, a UDG domain, a UGI domain, a methylase, a demethylase, a histone deacetylase, a DNA ligase, an epitope tag, and a reporter protein;

optionally, the subcellular localization signal is selected from: a nuclear localization signal, a nuclear export signal, a mitochondrial localization signal, and a chloroplast localization signal.

In the present disclosure, the fusion protein is preferably a fusion protein obtained by replacing amino acid residues 943-952 of a nuclease-inactivated mutant of a Cas9 protein having the amino acid sequence as set forth in SEQ ID NO: 1 with a functional domain;
optionally, the inactivated mutant has D10A and N863A mutations relative to SEQ ID NO: 1, or D10A and H840A mutations;
optionally, the functional domain is a FokI domain; further, the FokI domain is the DNA cleavage domain of the FokI restriction endonuclease; still further, the amino acid sequence of the FokI domain is as set forth in SEQ ID NO: 52;
optionally, the functional domain is connected to the N-terminus and C-terminus of the Cas9 inactivated mutant, respectively, through linker sequences; further, the functional domain is connected to the N-terminus and C-terminus of the Cas9 inactivated mutant, respectively, through linkers having the amino acid sequences as set forth in SEQ ID NO: 50 and SEQ ID NO: 51;
optionally, at least one nuclear localization sequence is fused to the N-terminus and/or C-terminus of the fusion protein;
optionally, the fusion protein is capable of forming a complex with a guide polynucleotide, wherein the guide polynucleotide comprises a guide sequence, and the guide sequence is engineered to direct the complex to bind to double-stranded target DNA in a sequence-specific manner and nick the target DNA; further, the guide polynucleotide is a single-molecule guide polynucleotide or a dual-molecule guide polynucleotide; further, the guide polynucleotide is a single-molecule guide polynucleotide formed by chimerizing crRNA and tracrRNA in a CRISPR-Cas9 system, or the guide polynucleotide is a set composed of two separate molecules, crRNA and tracrRNA, in a CRISPR-Cas9 system, wherein the crRNA consists of a guide sequence and a direct repeat sequence.

In some embodiments of the present disclosure, the fusion protein comprises the amino acid sequence as set forth in SEQ ID NO: 3.

In some specific embodiments of the present disclosure, the amino acid sequence of the fusion protein is as set forth in SEQ ID NO: 3.

In a second aspect, the present disclosure provides a ribonucleoprotein complex (RNP complex), wherein the RNP complex comprises a guide polynucleotide and the fusion protein according to the first aspect.

In some embodiments of the present disclosure, the guide polynucleotide directs the ribonucleoprotein complex to bind to a target nucleic acid in a sequence-specific manner.

In some embodiments of the present disclosure, the guide polynucleotide comprises a guide sequence, and the guide sequence is engineered to direct the ribonucleoprotein complex to bind to a target nucleic acid in a sequence-specific manner.

In some preferred embodiments of the present disclosure, the guide polynucleotide further comprises a scaffold sequence; the scaffold sequence interacts with the dCas9 portion of the fusion protein, or the scaffold sequence enables the guide polynucleotide to form a complex with the fusion protein.

In some embodiments of the present disclosure, the guide polynucleotide is a single-molecule guide polynucleotide or a dual-molecule guide polynucleotide.

In a third aspect, the present disclosure provides a gene editing system, wherein the gene editing system comprises:
(1) the fusion protein according to the first aspect, or a nucleic acid encoding the same; and/or
(2) a guide polynucleotide, or a nucleic acid encoding the same.

In some embodiments of the present disclosure, the guide polynucleotide directs the fusion protein to bind to a target nucleic acid in a sequence-specific manner.

In some embodiments of the present disclosure, the guide polynucleotide comprises a guide sequence, and the guide sequence is engineered to direct the ribonucleoprotein complex to bind to a target nucleic acid in a sequence-specific manner.

In some preferred embodiments of the present disclosure, the guide polynucleotide further comprises a scaffold sequence; the scaffold sequence enables the guide polynucleotide to form a complex with the fusion protein.

In some embodiments of the present disclosure, the gene editing system comprises 1, 2, 3, 4, 5, 6, 7, 8, 9, 10, or more different fusion proteins. In some embodiments of the present disclosure, the gene editing system comprises 1 fusion protein.

In some embodiments of the present disclosure, the gene editing system comprises at least 2 different guide polynucleotides.

In some embodiments of the present disclosure, the gene editing system comprises 1, 2, 3, 4, 5, 6, 7, 8, 9, 10, or more different guide polynucleotides. In some embodiments of the present disclosure, the gene editing system comprises 2 different guide polynucleotides.

In some embodiments of the present disclosure, the different guide polynucleotides have different guide sequences and the same scaffold sequence. In some embodiments of the present disclosure, the different guide polynucleotides have different guide sequences and different scaffold sequences.

In some embodiments of the present disclosure, the guide polynucleotide is a single-molecule guide polynucleotide or a dual-molecule guide polynucleotide. In some embodiments of the present disclosure, the guide polynucleotide is a single-molecule guide polynucleotide obtained by chimerizing crRNA and tracrRNA. In some embodiments of the present disclosure, the guide polynucleotide is a single-molecule guide polynucleotide obtained by chimerizing crRNA and tracrRNA through a nucleotide sequence. In some embodiments of the present disclosure, the guide polynucleotide is a dual-molecule guide polynucleotide composed of a crRNA molecule and a tracrRNA molecule.

In a fourth aspect, the present disclosure provides a polynucleotide, wherein the polynucleotide encodes the fusion protein according to the first aspect or the ribonucleoprotein complex according to the second aspect.

In some embodiments of the present disclosure, in the polynucleotide encoding the ribonucleoprotein complex, the polynucleotide encoding the guide polynucleotide and the polynucleotide encoding the fusion protein are on the same or different nucleic acid strands.

In a fifth aspect, the present disclosure provides an expression cassette, wherein the expression cassette comprises a promoter and the polynucleotide according to the fourth aspect; the promoter sequence regulates expression of the coding sequence.

In a sixth aspect, the present disclosure provides a recombinant expression vector, wherein the recombinant expression vector comprises the polynucleotide according to the fourth aspect, and optionally a cis-acting element and/or a trans-acting factor-encoding gene.

In some embodiments of the present disclosure, the cis-acting element is selected from a promoter, an enhancer, and a silencer.

In some embodiments of the present disclosure, the trans-acting factor-encoding gene is selected from nucleotides encoding a polymerase, a transcription factor, and/or a transcriptional regulatory factor.

In a seventh aspect, the present disclosure provides a composition, wherein the composition comprises a delivery vehicle, and the fusion protein according to the first aspect, the ribonucleoprotein complex according to the second aspect, the gene editing system according to the third aspect, the polynucleotide according to the fourth aspect, the expression cassette according to the fifth aspect, and/or the recombinant expression vector according to the sixth aspect.

In an eighth aspect, the present disclosure provides a recombinant cell, wherein the recombinant cell comprises the fusion protein according to the first aspect, the ribonucleoprotein complex according to the second aspect, the gene editing system according to the third aspect, the polynucleotide according to the fourth aspect, the expression cassette according to the fifth aspect, the recombinant expression vector according to the sixth aspect, and/or the composition according to the seventh aspect.

In a ninth aspect, the present disclosure provides a pharmaceutical composition, wherein the pharmaceutical composition comprises the fusion protein according to the first aspect, the ribonucleoprotein complex according to the second aspect, the gene editing system according to the third aspect, the polynucleotide according to the fourth aspect, the expression cassette according to the fifth aspect, the recombinant expression vector according to the sixth aspect, the composition according to the seventh aspect, and/or the recombinant cell according to the eighth aspect, and optionally a pharmaceutically acceptable carrier and/or excipient.

In a tenth aspect, the present disclosure provides a kit, wherein the kit comprises the fusion protein according to the first aspect, the ribonucleoprotein complex according to the second aspect, the gene editing system according to the third aspect, the polynucleotide according to the fourth aspect, the expression cassette according to the fifth aspect, the recombinant expression vector according to the sixth aspect, the composition according to the seventh aspect, the recombinant cell according to the eighth aspect, and/or the pharmaceutical composition according to the ninth aspect.

In an eleventh aspect, the present disclosure provides use of the fusion protein according to the first aspect, the ribonucleoprotein complex according to the second aspect, the gene editing system according to the third aspect, the polynucleotide according to the fourth aspect, the expression cassette according to the fifth aspect, the recombinant expression vector according to the sixth aspect, the composition according to the seventh aspect, the recombinant cell according to the eighth aspect, the pharmaceutical composition according to the ninth aspect, and/or the kit according to the tenth aspect in the preparation of a medicament for diagnosing, preventing, and/or treating a disease or disorder related to a target nucleic acid.

In a twelfth aspect, the present disclosure provides a method for diagnosing, preventing, and/or treating a disease or disorder related to a target nucleic acid, wherein the method comprises administering to a patient in need thereof the fusion protein according to the first aspect, the ribonucleoprotein complex according to the second aspect, the gene editing system according to the third aspect, the polynucleotide according to the fourth aspect, the expression cassette according to the fifth aspect, the recombinant expression vector according to the sixth aspect, the composition according to the seventh aspect, the recombinant cell according to the eighth aspect, the pharmaceutical composition according to the ninth aspect, and/or the kit according to the tenth aspect.

In a thirteenth aspect, the present disclosure provides the fusion protein according to the first aspect, the ribonucleoprotein complex according to the second aspect, the gene editing system according to the third aspect, the polynucleotide according to the fourth aspect, the expression cassette according to the fifth aspect, the recombinant expression vector according to the sixth aspect, the composition according to the seventh aspect, the recombinant cell according to the eighth aspect, the pharmaceutical composition according to the ninth aspect, and/or the kit according to the tenth aspect, wherein the fusion protein, ribonucleoprotein complex, gene editing system, polynucleotide, expression cassette, recombinant expression vector, composition, recombinant cell, pharmaceutical composition, or kit is for use in diagnosing, preventing, and/or treating a disease or disorder related to a target nucleic acid.

In a fourteenth aspect, the present disclosure provides a method for editing a target nucleic acid in vitro, ex vivo, or in vivo, wherein the method comprises contacting the target nucleic acid with the fusion protein according to the first aspect, the ribonucleoprotein complex according to the second aspect, the gene editing system according to the third aspect, the polynucleotide according to the fourth aspect, the expression cassette according to the fifth aspect, the recombinant expression vector according to the sixth aspect, the composition according to the seventh aspect, the recombinant cell according to the eighth aspect, the pharmaceutical composition according to the ninth aspect, and/or the kit according to the tenth aspect, thereby changing the sequence of the target nucleic acid or altering the expression level of the target nucleic acid.

In one example of the present disclosure, the method comprises contacting a double-stranded target nucleic acid with the first fusion protein, second fusion protein, first guide polynucleotide, and second guide polynucleotide according to the present disclosure, thereby changing the sequence of the target nucleic acid or altering the expression level of the target nucleic acid;

wherein the first fusion protein can bind, together with the first guide polynucleotide, to one single strand of the double-stranded target nucleic acid, and the second fusion protein can bind, together with the second guide polynucleotide, to the other single strand of the target nucleic acid;

the FokI domains of the first fusion protein and the second fusion protein are separated by a spacer sequence on the double-stranded target nucleic acid and can form a dimer, the spacer sequence having a length of 1-100 bp, and the dimer can cleave the double-stranded target nucleic acid when the first fusion protein, the first guide polynucleotide, the second fusion protein, and the second guide polynucleotide bind to the target nucleic acid.

On the basis of common general knowledge in the art, the above preferred conditions can be combined arbitrarily to obtain the preferred examples of the present disclosure.

The beneficial advances of the present disclosure are as follows: the gene editing system containing the fusion protein of the present disclosure has high editing efficiency and/or low off-target risk.

### BRIEF DESCRIPTION OF THE DRAWINGS

FIG. 1 is a schematic diagram showing replacement within a dCas9 protein with a FokI domain having single-stranded nucleic acid nicking capability.

FIG. 2 is a schematic diagram showing that dual gRNAs guide dCas9-FokI to target and cleave two single strands, thereby forming a double-strand break;

the nucleotide sequence in the figure is as set forth in SEQ ID NO: 32, and the amino acid sequence is as set forth in SEQ ID NO: 33.

FIG. 3 is a schematic diagram of flow cytometric detection results after editing by the dCas9-FokI fusion protein.

FIG. 4 is a schematic diagram of the editing efficiencies of different recombinant proteins targeting a reporter system.

FIG. 5 is a schematic diagram of the editing efficiencies of different recombinant proteins targeting endogenous genes.

### DETAILED DESCRIPTION

The present disclosure is further described below by way of examples, but the present disclosure is not thereby limited to the scope of the described examples. Experimental methods for which specific conditions are not indicated in the following examples are carried out according to conventional methods and conditions, or selected according to product instructions.

### Terms

In the present disclosure, unless otherwise specified, the scientific and technical terms used herein have the meanings commonly understood by those skilled in the art. Moreover, the procedures for molecular genetics, nucleic acid chemistry, chemistry, molecular biology, biochemistry, cell culture, microbiology, cell biology, genomics, recombinant DNA, and the like used herein are conventional procedures widely used in the corresponding fields. At the same time, for a better understanding of the present disclosure, relevant terms are defined and explained below.

In the present disclosure, letters in amino acid sequences represent the one-letter abbreviations of amino acids known in the art, for example, as described in J. Biol. Chem., 243, p3558 (1968): alanine: Ala-A, arginine: Arg-R, aspartic acid: Asp-D, cysteine: Cys-C, glutamine: Gln-Q, glutamic acid: Glu-E, histidine: His-H, glycine: Gly-G, asparagine: Asn-N, tyrosine: Tyr-Y, proline: Pro-P, serine: Ser-S, methionine: Met-M, lysine: Lys-K, valine: Val-V, isoleucine: Ile-I, phenylalanine: Phe-F, leucine: Leu-L, tryptophan: Trp-W, and threonine: Thr-T.

In the present disclosure, taking the SpCas9 protein as an example, the number represented by a position refers to the position of the amino acid residue corresponding to the amino acid sequence SEQ ID NO: 1 of the wild-type SpCas9 protein. In the present disclosure, the letter before a position indicates the original amino acid residue, and the letter after the position indicates the substituted amino acid residue.

In the present disclosure, the term “identity” (or “percent identity”) is used to refer to the degree of sequence match between two polypeptides or between two nucleic acids. When a certain position in each of two sequences being compared is occupied by the same base or amino acid monomer subunit (for example, a certain position in each of two DNA molecules is occupied by adenine, or a certain position in each of two polypeptides is occupied by lysine), the molecules are identical at that position. “Percent sequence identity” between two sequences is a function of the number of matching positions shared by the two sequences divided by the number of positions compared×100%. For example, if 6 out of 10 positions of two sequences match, the two sequences have 60% sequence identity. Typically, comparisons are made by aligning two sequences to produce the maximum sequence identity. Such alignment may be performed using published and commercially available alignment algorithms and programs, including but not limited to Clustal Q, MAFFT, Probcons, T-Coffee, Probalign, and BLAST, as may be reasonably selected by those of ordinary skill in the art. Those skilled in the art can determine suitable parameters for sequence alignment, including any algorithm needed to achieve an optimal alignment or best comparison over the full length of the compared sequences, as well as any algorithm needed to achieve an optimal alignment or best comparison over local regions of the compared sequences.

In the present disclosure, sequence comparison and determination of percent identity between two sequences may be implemented using mathematical algorithms. In some cases, percent identity between two amino acid sequences is determined using the Needleman and Wunsch ((1970) J. Mol. Biol. 48:444-453) algorithm, with the Blossum 62 scoring matrix, a gap penalty of 12, a gap extension penalty of 4, and a frameshift gap penalty of 5, which algorithm is incorporated into the GAP program in the GCG software package.

### Cas9 Protein and Gene Editing

Cas9 protein is a common Cas protein in CRISPR type II systems. Some wild-type Cas9 proteins have been detected to have nuclease activity and are capable of cleaving nucleic acids or nucleic acid strands. The Cas9 protein is usually capable of forming an RNP complex with a guide polynucleotide and being guided by the guide polynucleotide to a cleavage site.

Cas9 molecules from many species can be used in the fusion proteins described herein. Although Streptococcus pyogenes and Staphylococcus aureus Cas9 molecules are the subject of much of the disclosure herein, Cas9 molecules of other species listed herein, and Cas9 molecules derived from or based on the Cas9 proteins of such species, may likewise be used. In other words, although much of the description herein uses Streptococcus pyogenes and Staphylococcus aureus Cas9 molecules, Cas9 molecules from other species may be used in place thereof.

Gene editing refers to editing of a single base or a nucleic acid strand based on various gene editing tools, thereby altering the original nucleic acid at the molecular level. Accordingly, gene editing includes single-base editing and editing of nucleic acid strands. As used herein, the term “single base” refers to one and only one nucleotide within a nucleic acid sequence. When used in the context of single-base editing, it refers to replacement of a base at a specific position within a nucleic acid sequence with a different base. Such replacement can occur by many mechanisms, including but not limited to substitution or modification.

As used herein, the term “nicking” refers to breaking internucleotide or interstrand bonds in a nucleic acid strand, thereby generating a gap in a single-stranded or double-stranded nucleic acid. The term “cleaving” refers to breaking at least interstrand bonds in a double-stranded nucleic acid, or at least internucleotide bonds in a single-stranded nucleic acid, such that nucleic acid strands upstream and downstream of the cleavage site are completely separated.

### FokI Domain

FokI is a type II restriction endonuclease comprising a DNA recognition domain and a catalytic (endonuclease) domain. The fusion proteins described herein may comprise full-length FokI, or only the nuclease domain of FokI, for example amino acids 388-583 or 408-583 of GenBank accession number AAA24927.1, such as the mutant forms of FokI described in, for example, Li et al., Nucleic Acids Res. 39 (1): 359-372 (2011); Cathomen and Joung, Mol. Ther. 16:1200-1207 (2008); Miller et al., Nat. Biotechnol. 25:778-785 (2007); Szczepek et al., Nat. Biotechnol. 25:786-793 (2007); or Bitinaite et al., Proc. Natl. Acad. Sci. USA. 95:10570-10575 (1998).

The FokI domain may be the DNA cleavage domain of a wild-type FokI restriction endonuclease; the FokI domain may also be a mutant of the DNA cleavage domain of a wild-type FokI restriction endonuclease, including but not limited to a single-point mutant and a multi-point mutant. In some embodiments, two FokI domains can form a dimer. In some embodiments, two FokI domains cannot form a dimer. In some embodiments, the two FokI domains acquire nuclease activity only after dimerization. In some embodiments, the two FokI domains can cleave a target nucleic acid only after dimerization. In some embodiments, the FokI domain can cleave a target nucleic acid without requiring dimerization. In some embodiments, the FokI domain has nuclease activity without requiring dimerization.

### Guide Polynucleotide

As used herein, the term “guide polynucleotide” refers to a molecule that forms a CRISPR complex with a Cas protein in a CRISPR-Cas system and guides the CRISPR complex to a target nucleic acid. Generally, the guide polynucleotide comprises a scaffold sequence linked to a guide sequence, and the guide sequence can hybridize to the target nucleic acid sequence. The scaffold sequence generally comprises a direct repeat sequence and may sometimes further comprise a tracrRNA sequence. The direct repeat sequence and the guide sequence together constitute a crRNA.

In some embodiments, the guide polynucleotide is a guide RNA. In some embodiments, the guide polynucleotide is a guide DNA. In some embodiments, at least one base of the guide polynucleotide is a DNA base. In some embodiments, the guide polynucleotide is a chemically modified guide polynucleotide. In some embodiments, the guide polynucleotide comprises at least one chemically modified nucleotide.

In some embodiments, the guide polynucleotide comprises at least one guide sequence (guide sequence, also referred to as a spacer sequence) linked to at least one direct repeat sequence (direct repeat, DR). In some embodiments, the guide sequence is located at the 3′ end of the scaffold sequence. In some embodiments, the guide sequence is located at the 5′ end of the scaffold sequence.

In some embodiments, the direct repeat sequence comprises at least 5, at least 10, at least 15, at least 20, at least 25, at least 30, at least 35, at least 40, at least 45, at least 50, at least 55, or at least 60 nucleotides. In some embodiments, the direct repeat sequence comprises no more than 70 nucleotides, no more than 60 nucleotides, no more than 55 nucleotides, no more than 50 nucleotides, no more than 45 nucleotides, no more than 40 nucleotides, no more than 35 nucleotides, no more than 30 nucleotides, no more than 25 nucleotides, no more than 20 nucleotides, or no more than 15 nucleotides. In some embodiments, the direct repeat sequence comprises 5-70 nucleotides, 5-50 nucleotides, 5-30 nucleotides, 5-20 nucleotides, 5-15 nucleotides, 10-40 nucleotides, 10-30 nucleotides, 10-20 nucleotides, or 10-15 nucleotides.

In some embodiments, the tracrRNA sequence comprises at least 5, at least 10, at least 15, at least 20, at least 25, at least 30, at least 35, at least 40, at least 45, at least 50, at least 55, at least 60, at least 65, at least 70, at least 75, or at least 80 nucleotides. In some embodiments, the tracrRNA sequence comprises no more than 120 nucleotides, no more than 110 nucleotides, no more than 100 nucleotides, no more than 90 nucleotides, no more than 80 nucleotides, no more than 70 nucleotides, no more than 60 nucleotides, no more than 55 nucleotides, no more than 50 nucleotides, no more than 45 nucleotides, no more than 40 nucleotides, no more than 35 nucleotides, no more than 30 nucleotides, no more than 25 nucleotides, no more than 20 nucleotides, or no more than 15 nucleotides. In some embodiments, the direct repeat sequence comprises 10-120 nucleotides, 20-100 nucleotides, 30-90 nucleotides, 40-80 nucleotides, 40-70 nucleotides, 50-70 nucleotides, or 60-70 nucleotides.

In some embodiments, the guide sequence comprises at least 15, at least 16, at least 17, at least 18, at least 19, at least 20, at least 21, at least 22, at least 23, at least 24, at least 25, at least 26, at least 27, at least 28, at least 29, or at least 30 nucleotides. In some embodiments, the guide sequence comprises no more than 60 nucleotides, no more than 55 nucleotides, no more than 50 nucleotides, no more than 45 nucleotides, no more than 40 nucleotides, no more than 35 nucleotides, no more than 30 nucleotides, no more than 25 nucleotides, or no more than 20 nucleotides. In some embodiments, the guide sequence comprises 15-60 nucleotides, 15-40 nucleotides, 15-30 nucleotides, 15-25 nucleotides, or 17-25 nucleotides.

In some embodiments, the guide sequence has sufficient complementarity to the target nucleic acid to hybridize with the target nucleic acid and direct the fusion protein of the present disclosure to bind to the target nucleic acid in a sequence-specific manner. In some embodiments, the guide sequence has 100% complementarity to the target nucleic acid, but the guide sequence may have less than 100% complementarity to the target nucleic acid DNA, for example at least 80%, at least 85%, at least 90%, at least 95%, at least 98%, or at least 99% complementarity.

In some embodiments, the guide sequence is engineered to hybridize to the target nucleic acid with no more than two nucleotide mismatches. In some embodiments, the guide sequence is engineered to hybridize to the target nucleic acid with no more than one nucleotide mismatch. In some embodiments, the guide sequence is engineered to hybridize to the target nucleic acid with or without a mismatch.

The guide polynucleotide may be a single molecule or a dual molecule. The guide polynucleotide sequence may be an RNA sequence, a DNA sequence, or a combination thereof (an RNA-DNA combined sequence). Optionally, the guide polynucleotide may comprise at least one nucleotide, phosphodiester bond, or conjugation modification, including but not limited to locked nucleic acid (LNA), 5-methyl dC, 2,6-diaminopurine, 2′-fluoro A, 2′-fluoro U, 2′-O-methyl RNA, a phosphorothioate bond, conjugation to a cholesterol molecule, conjugation to a polyethylene glycol molecule, conjugation to a spacer 18 (hexaethylene glycol chain) molecule, or a 5′-to-3′ covalent linkage resulting in cyclization.

The guide polynucleotide may be a single-molecule guide polynucleotide or a dual-molecule guide polynucleotide. By way of non-limiting example, the guide polynucleotide is a single-molecule guide polynucleotide (sgRNA) formed by chimerizing crRNA and tracrRNA in a CRISPR-Cas9 system (for example, through a GAAA sequence). By way of another example, the guide polynucleotide is a set composed of two separate molecules, crRNA and tracrRNA, in the CRISPR-Cas9 system, namely the dual-molecule form.

The ability of the guide polynucleotide to direct a ribonucleoprotein complex to bind to target DNA in a sequence-specific manner can be assessed by any suitable assay. For example, components of a CRISPR system sufficient to form a ribonucleoprotein complex, including the guide polynucleotide to be tested, can be provided to a host cell having a corresponding target nucleic acid DNA molecule, for example by transfection of a vector encoding the components of the ribonucleoprotein complex, and preferential cleavage within the target sequence can then be evaluated. Similarly, cleavage of a target nucleic acid DNA sequence can be assessed in vitro by providing target nucleic acid DNA, components of the ribonucleoprotein complex including the guide polynucleotide to be tested, and a control guide polynucleotide different from the test guide polynucleotide, and comparing the ability of the test and control guide polynucleotides to bind to the target nucleic acid DNA or the rate of cleavage of the target DNA. The ability of the ribonucleoprotein complex to cleave a target nucleic acid or target DNA can also be evaluated by the above assays.

### Gene Editing System

The gene editing system of the present disclosure includes, but is not limited to, systems used for the following purposes: cleaving a target nucleic acid, introducing a base conversion into a target nucleic acid, suppressing or reducing expression of a specific gene on a target nucleic acid, activating or increasing expression of a specific gene on a target nucleic acid, visualizing or detecting a target nucleic acid, masking a specific gene on a target nucleic acid, and silencing a specific gene on a target nucleic acid.

### Vector System

Another aspect of the present disclosure relates to a vector system comprising the fusion protein described herein, wherein the vector system comprises one or more vectors (expression vectors), and the vector(s) comprise a polynucleotide sequence encoding the fusion protein and a polynucleotide sequence encoding the guide polynucleotide. Alternatively, the vector comprises a polynucleotide sequence encoding the fusion protein.

In some embodiments, the vector system comprises at least one plasmid or viral vector (for example, a retrovirus, lentivirus, adenovirus, adeno-associated virus, or herpes simplex virus). In some embodiments, the polynucleotide sequence encoding the fusion protein and the polynucleotide sequence encoding the guide polynucleotide are located on the same vector. In some embodiments, the polynucleotide sequence encoding the fusion protein and the polynucleotide sequence encoding the guide polynucleotide are located on 2 or more vectors.

In some embodiments, the polynucleotide sequence encoding the fusion protein and/or the polynucleotide sequence encoding the guide polynucleotide is operably linked to regulatory elements. Regulatory elements include promoters, enhancers, internal ribosome entry sites (IRES), and other expression control elements (for example, transcription termination signals such as polyadenylation signals and poly-U sequences). Regulatory elements include elements that direct constitutive expression of a nucleotide sequence in many types of host cells, as well as elements that direct expression of the nucleotide sequence only in certain host cells (for example, tissue-specific regulatory sequences). Tissue-specific promoters can direct expression primarily in the desired tissue of interest, for example muscle, neurons, bone, skin, blood, a specific organ (for example, liver or pancreas), or a specific cell type (for example, lymphocytes). Regulatory elements may also direct expression in a time-dependent manner, for example in a cell cycle-dependent or developmental stage-dependent manner, which may or may not also be tissue- or cell type-specific. In some embodiments, the regulatory element is an enhancer element, for example WPRE, a CMV enhancer, the R-U5 segment in the LTR of HTLV-1, an SV40 enhancer, or the intron sequence between rabbit β-globin exons 2 and 3.

In some embodiments, the vector comprises a pol III promoter (for example, a U6 or H1 promoter), a pol II promoter (for example, a retroviral Rous sarcoma virus (RSV) LTR promoter, optionally with an RSV enhancer; a cytomegalovirus (CMV) promoter, optionally with a CMV enhancer; an SV40 promoter; a dihydrofolate reductase promoter; a β-actin promoter; a phosphoglycerate kinase (PGK) promoter; or an EF1a promoter), or both a pol III promoter and a pol II promoter.

In some embodiments, the promoter is a constitutive promoter, which is continuously active and is not regulated by external signals or molecules. Suitable constitutive promoters include, but are not limited to, CMV, RSV, SV40, EF1α, CAG, and β-actin promoters. In some embodiments, the promoter is an inducible promoter regulated by an external signal or molecule (for example, a transcription factor).

In some embodiments, the promoter is a tissue-specific promoter, which can be used to drive tissue-specific expression of the fusion protein. Suitable muscle-specific promoters include, but are not limited to, CK8, MHCK7, a myoglobin promoter (Mb), a desmin promoter, a muscle creatine kinase promoter (MCK) and variants thereof, and the synthetic promoter SPc5-12. Suitable immune cell-specific promoters include, but are not limited to, the B29 promoter (B cells), the CD14 promoter (monocytes), the CD43 promoter (leukocytes and platelets), CD68 (macrophages), and the SV40/CD43 promoter (leukocytes and platelets). Suitable blood cell-specific promoters include, but are not limited to, the CD43 promoter (leukocytes and platelets), the CD45 promoter (hematopoietic cells), INF-β (hematopoietic cells), the WASP promoter (hematopoietic cells), the SV40/CD43 promoter (leukocytes and platelets), and the SV40/CD45 promoter (hematopoietic cells). Suitable pancreas-specific promoters include, but are not limited to, the elastase-1 promoter. Suitable endothelial cell-specific promoters include, but are not limited to, the Fit-1 promoter and the ICAM-2 promoter. Suitable neuronal tissue/cell-specific promoters include, but are not limited to, the GFAP promoter (astrocytes), the SYN1 promoter (neurons), and NSE/RU5′ (mature neurons). Suitable kidney-specific promoters include, but are not limited to, the NphsI promoter (podocytes). Suitable bone-specific promoters include, but are not limited to, the OG-2 promoter (osteoblasts, odontoblasts). Suitable lung-specific promoters include, but are not limited to, the SP-B promoter (lung). Suitable liver-specific promoters include, but are not limited to, the SV40/Alb promoter. Suitable cardiac-specific promoters include, but are not limited to, α-MHC.

### AAV Vector

Another aspect of the present disclosure relates to an adeno-associated virus (AAV) vector comprising the fusion protein or gene editing system described herein, wherein the adeno-associated virus (AAV) vector comprises DNA encoding the fusion protein and guide polynucleotide described herein.

Delivery of a CRISPR-Cas system via an AAV vector is described in Maeder et al., Nature Medicine 25:229-233 (2019), wherein subretinal delivery of AAV has been clinically demonstrated to be safe and effective, and local delivery by subretinal injection, the natural tropism of AAV5 for photoreceptor cells, and the use of a photoreceptor-specific GRK1 promoter are used to confine expression of the CRISPR/Cas system to therapeutic target tissues and cell types, the entirety of which is incorporated herein by reference. In some embodiments, the AAV vector comprises an ssDNA genome containing coding sequences for the fusion protein and guide polynucleotide flanked by ITRs.

In some embodiments, the fusion protein or gene editing system described herein is packaged in an AAV vector, for example AAV1, AAV2, AAV3, AAV4, AAV5, AAV6, AAV7, AAV8, AAV9, or AAVrh74. In some embodiments, the fusion protein or gene editing system described herein is packaged in an AAV vector comprising an engineered capsid having tissue tropism, for example an engineered muscle-tropic capsid. Tabebordbar et al., Cell 184:4919-4938 (2021) describes engineering of AAV capsids having tissue tropism by directed evolution, identifying a class of capsids containing an RGD motif, wherein systemic injection of MyoAAV efficiently transduces primate muscle, the entirety of which is incorporated herein by reference.

### Lipid Nanoparticle

Another aspect of the present disclosure relates to a lipid nanoparticle (LNP) comprising the gene editing system described herein, wherein the LNP comprises the guide polynucleotide described herein and mRNA encoding the fusion protein described herein.

Gillmore et al., N. Engl. J. Med., 385:493-502 (2021) describes LNP delivery of a CRISPR-Cas system, wherein the lipid nanoparticle (LNP) consists of 4 lipids, including the proprietary ionizable lipid LP000001, DSPC, cholesterol, and DMG-PEG2k, and the LNP suspension is formulated in an aqueous buffer of Tris, NaCl, and sucrose at pH 7.4, the entirety of which is incorporated herein by reference. In some embodiments, in addition to the RNA payload (mRNA and guide polynucleotide), the lipid nanoparticle (LNP) further comprises four components: a cationic or ionizable lipid, cholesterol, a helper lipid, and a PEG-lipid. In some embodiments, the cationic or ionizable lipid includes CKK-E12, C12-200, ALC-0315, DLin-MC3-DMA, DLin-KC2-DMA, FTT5, Moderna SM-102, and Intellia LP01. In some embodiments, the PEG-lipid comprises PEG-2000-C-DMG, PEG-2000-DMG, or ALC-0159. In some embodiments, the helper lipid comprises DSPC. Components of LNPs are described in Paunovska et al., Nature Reviews Genetics 23:265-280 (2022), wherein FDA-approved LNPs comprise variants of four basic components: a cationic or ionizable lipid, cholesterol, a helper lipid, and a polyethylene glycol (PEG) lipid, the entirety of which is incorporated herein by reference.

### Lentiviral Vector

Another aspect of the present disclosure relates to a lentiviral vector comprising the gene editing system described herein, wherein the lentiviral vector comprises the guide polynucleotide described herein and mRNA encoding the fusion protein described herein. In some embodiments, the lentiviral vector is pseudotyped with a homologous or heterologous envelope protein such as VSV-G. In some embodiments, the mRNA encoding the fusion protein is linked to an aptamer sequence.

### RNP Complex

Another aspect of the present disclosure relates to a ribonucleoprotein complex described herein, wherein the ribonucleoprotein complex is formed from the guide polynucleotide and fusion protein described herein. In some embodiments, the ribonucleoprotein complex may be delivered into a eukaryotic cell, mammalian cell, or human cell by microinjection or electroporation. In some embodiments, the ribonucleoprotein complex may be packaged in virus-like particles and delivered in vivo to a mammalian or human subject.

### Virus-Like Particle

Another aspect of the present disclosure relates to a virus-like particle (VLP) comprising the gene editing system described herein, wherein the virus-like particle comprises the guide polynucleotide and fusion protein described herein, or a ribonucleoprotein complex composed of the guide polynucleotide and fusion protein.

Banskota et al., Cell 185 (2): 250-265 (2022) reported the development and application of DNA-free engineered virus-like particles (eVLPs) for efficient packaging and delivery of base editors or Cas9 ribonucleoproteins; Mangeot et al., Nature Communications 10 (1): 1-15 (2019) used engineered murine leukemia virus-like particles (Nanoblades) loaded with Cas9-sgRNA ribonucleoproteins to induce efficient genome editing in cell lines and primary cells, including human induced pluripotent stem cells, human hematopoietic stem cells, and mouse bone marrow cells; Campbell et al., Molecular Therapy 27:151-163 (2019) utilized specialized extracellular vesicles known as “gesicles” to effectively but transiently deliver, in ribonucleoprotein form, Cas9 targeting the HIV long terminal repeat sequence (LTR), wherein gesicles are generated by expressing vesicular stomatitis virus glycoprotein and packaging proteins as cargo, thereby obviating transgenic delivery and allowing finer control of Cas9 expression; and Mangeot et al., Molecular Therapy 19 (9): 1656-1666 (2011) reported that overexpression of the spike glycoprotein of vesicular stomatitis virus (VSV-G) in human cells induced release of fusogenic vesicles termed gesicles, and biochemical and functional studies showed that the vesicles incorporated proteins from producer cells and could deliver them to recipient cells, such protein transduction allowing direct transport of cytoplasmic, nuclear, or surface proteins in target cells. These references all describe engineered VLPs, and the entirety of each is incorporated herein by reference.

In some embodiments, the engineered virus-like particle (VLP) is pseudotyped with a homologous or heterologous envelope protein such as VSV-G. In some embodiments, the fusion protein is fused to a gag protein (for example, MLV gag) via a cleavable linker, wherein cleavage of the linker in the target cell exposes an NLS located between the linker and the fusion protein. In some embodiments, the fusion protein or conjugate comprises (for example, from 5′ to 3′) a gag protein (for example, MLV gag), one or more NESs, a cleavable linker, one or more NLSs, and Cas12, as described in Banskota et al., Cell 185 (2): 250-265 (2022).

In some embodiments, the fusion protein is fused to a first dimerization domain, and the first dimerization domain is capable of dimerizing or heterodimerizing with a second dimerization domain fused to a membrane protein, wherein the presence of a ligand promotes the dimerization and enriches the Cas12 protein or fusion protein or conjugate into the VLP, as described in Campbell et al., Molecular Therapy 27:151-163 (2019).

### Cell

Another aspect of the present disclosure relates to a cell comprising the fusion protein described herein and a gene editing system comprising the same. The cell (for example, which may be used to generate a cell-free system) may be eukaryotic or prokaryotic. Examples of such cells include, but are not limited to, bacteria, archaea, plants, fungi, yeast, insects, and mammalian cells, such as Lactobacillus, Lactococcus, Bacillus (for example, Bacillus subtilis), Escherichia (for example, Escherichia coli), Clostridium, Saccharomyces or Pichia (such as Saccharomyces cerevisiae or Pichia pastoris), Kluyveromyces lactis, Salmonella typhimurium, Drosophila cells, Caenorhabditis elegans cells, Xenopus laevis cells, SF9 cells, C129 cells, 293 cells, Neurospora, and immortalized mammalian cell lines (for example, Hela cells, myeloid cell lines, and lymphoid cell lines).

In some embodiments, the cell is a prokaryotic cell, for example a bacterial cell such as Escherichia coli. In some embodiments, the cell is a eukaryotic cell, for example a mammalian cell or a human cell. In some embodiments, the cell is a primary eukaryotic cell, a stem cell, a tumor/cancer cell, a circulating tumor cell (CTC), a blood cell (for example, a T cell, B cell, NK cell, Treg, or the like), a hematopoietic stem cell, a specialized immune cell (such as a tumor-infiltrating lymphocyte or tumor-suppressive lymphocyte), or a stromal cell in the tumor microenvironment (such as a cancer-associated fibroblast, or the like). In some embodiments, the cell is a brain or neuronal cell of the central or peripheral nervous system (for example, a neuron, astrocyte, microglial cell, retinal ganglion cell, rod cell, cone cell, or the like).

### Target Nucleic Acid or Target DNA

The fusion protein described herein and the gene editing system comprising the same can be used to target one or more target nucleic acids, such as target DNA molecules, for example target DNA molecules present in biological samples, environmental samples (for example, soil, air, or water samples), and the like.

In some embodiments, the target nucleic acid is a disease-related gene or a gene related to a signal transduction biochemical pathway, or the target nucleic acid is a reporter gene. Non-limiting examples of such target nucleic acids include those listed in U.S. Provisional Patent Applications 61/736,527 and 61/748,427 filed on Dec. 12, 2012 and Jan. 2, 2013, respectively, and International Application PCT/US2013/074667 filed on Dec. 12, 2013, all of which are incorporated herein by reference in their entireties.

### Therapeutic Applications

The present disclosure discloses compositions and pharmaceutical compositions comprising the fusion protein or gene editing system. In some embodiments, the pharmaceutical composition is delivered in vivo to a human subject. The pharmaceutical composition can be delivered by any effective route. Exemplary routes of administration include, but are not limited to, intravenous infusion, intravenous injection, intraperitoneal injection, intramuscular injection, intratumoral injection, subcutaneous injection, intradermal injection, intraventricular injection, intravascular injection, intracerebellar injection, intraocular injection, subretinal injection, intravitreal injection, intracameral injection, intratympanic injection, intranasal administration, and inhalation.

As used herein, the term “effective amount” refers to a specific amount of a pharmaceutical composition comprising a therapeutic agent that achieves a clinically beneficial result (that is, for example, alleviation of symptoms). The toxicity and therapeutic efficacy of such compositions can be determined by standard pharmaceutical procedures in cell culture or experimental animals, for example for determining the LD50 (the dose lethal to 50% of the population) and the ED50 (the dose therapeutically effective in 50% of the population). The dose ratio between toxicity and therapeutic effect is the therapeutic index, and it can be expressed as the ratio LD50/ED50. Compounds exhibiting high therapeutic indices are preferred. Data obtained from these cell culture assays and other animal studies can be used to formulate a range of dosages for human use. The dosage of such compounds preferably lies within a range of circulating concentrations that includes the ED50 with little or no toxicity. The dosage varies within this range depending on the dosage form employed, the sensitivity of the patient, and the route of administration.

As used herein, the term “disease” or “disorder” refers to any interruption of the normal state or impairment of the performance of vital functions in a living animal or plant body or a part thereof. It is generally manifested by characteristic signs and symptoms, and is generally a response to: i) environmental factors (such as malnutrition, industrial hazards, or climate); ii) specific infectious agents (such as helminths, bacteria, or viruses); iii) inherent defects of the organism (such as genetic abnormalities); and/or iv) a combination of these factors.

As used herein, the term “administration” or “administering” refers to any method of providing a composition to a patient such that the composition has an intended effect on the patient. Exemplary methods of administration are by direct means, such as local tissue administration (that is, for example, extravascular placement), oral administration, transdermal patches, topical administration, inhalation, suppositories, and the like.

As used herein, the term “patient” or “subject” is a human or animal and need not be hospitalized. For example, outpatients and persons in nursing homes are “patients.” Patients can include humans or non-human animals of any age and therefore include adults and minors (that is, children). The term “patient” does not imply that medical treatment is required; accordingly, a patient may voluntarily or involuntarily participate in an experiment, whether clinical or in support of basic scientific research.

### Example 1: Design of Different dCas9-FokI Fusion Proteins and Construction of Vector Plasmids

In order to insert the FokI nuclease domain into the interior of the dCas9 protein, possible insertion sites in the dCas9 protein were predicted and simulated by bioinformatics analysis and Al-based methods, and a FokI domain having single-stranded nucleic acid nicking activity was inserted or substituted on the premise that the DNA-binding capability of dCas9 would be unlikely to be affected. Specifically, random coil regions of SpCas9 were annotated in PyMOL, positions in the annotated random coil regions having a length greater than 3 aa were counted, and the corresponding domains were then annotated. Random coil sequences of dSpCas9 were replaced with the FokI domain. The first priority was to replace amino acid sequences in the RuvC region with the FokI domain. The second priority was that, the longer the random coil region, the smaller the potential impact of insertion of the FokI domain on the overall structure of SpCas9, and thus the higher the priority. On this basis, dCas9-FokI-S1 to dCas9-FokI-S10 fusion proteins were designed.

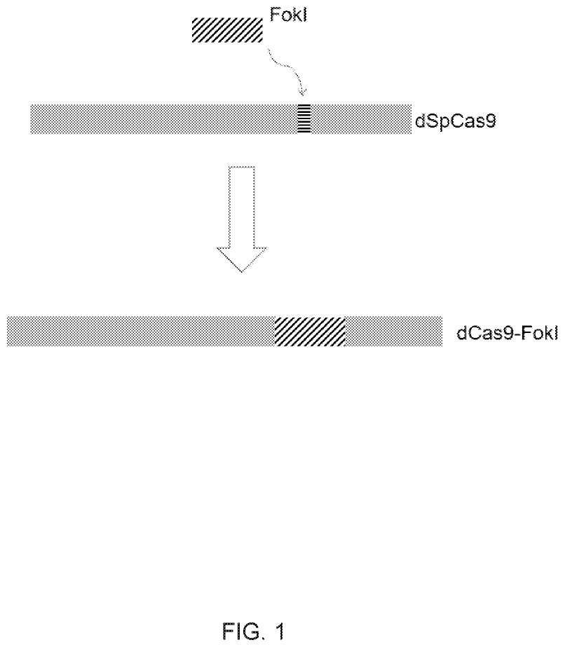

As shown in FIG. 1 and Table 1, vector plasmids for different dCas9-FokI fusion proteins were constructed using molecular biology methods.

In this example, the amino acid sequence of dCas9 was determined based on SpCas9 (SEQ ID NO: 1), as set forth in SEQ ID NO: 2.

TABLE 1
Designed Different dCas9-Fokl Fusion Proteins

| | | Length of Replaced | Domain in Which |
|---|---|---|---|
| | Replacement | Sequence (Number | the Replaced |
| | Position of | of Amino Acid | Sequence Is |
| Name | Fokl in SpCas9* | Residues) | Located** |
| dCas9- | N37 to H41 | 5 | RuvC-I |
| Fokl-S1 | | | |
| dCas9- | V107 to H113 | 7 | REC1-A |
| Fokl-S2 | | | |
| dCas9- | D147 to A149 | 3 | REC1-A |
| Fokl-S3 | | | |
| dCas9- | L169 to G172 | 4 | REC1-A |
| Fokl-S4 | | | |
| dCas9- | V308 to K314 | 7 | REC1-B |
| Fokl-S5 | | | |
| dCas9- | G792 to H799 | 8 | HNH |
| Fokl-S6 | | | |
| dCas9- | R864 to P871 | 8 | HNH |
| Fokl-S7 | | | |
| dCas9- | G906 to L908 | 3 | loop L2 |
| Fokl-S8 | | | |
| dCas9- | Y943 to E952 | 10 | RuvC-III |
| Fokl-S9 | | | |
| dCas9- | V1015 to D1017 | 3 | RuvC- III |
| Fokl-S10 | | | |

*indicates that the Fokl domain with a linker (SEQ ID NO: 4) was substituted at the indicated position; the Fokl domain (SEQ ID NO: 52) corresponds to residues 6-201 of the sequence set forth in SEQ ID NO: 4.
**Domain assignment of SpCas9 (SEQ ID NO: 1) refers to Zhu, X., et al. Cryo-EM structures reveal coordinated domain motions that govern DNA cleavage by Cas9. Nat Struct Mol Biol 26, 679-685 (2019). https://doi.org/10.1038/s41594-019-0258-2.

TABLE 2
List of Primers Corresponding to Different
dCas9-FokI Fusion Proteins

| | | SEQ |
|---|---|---|
| Primer | | ID |
| Name | Primer Sequence | NO |
| FokI-PF1 | GGAGGTTCTGGATCCCAACTAGTCAAAAG | 6 |
| FokI-PR1 | ACTTTCGGGTGTGGCGGACTCTGAG | 7 |
| dCas9-PF1 | CAAGCTGGCTAGCGTTTAAACTTAAGC | 8 |
| dCas9-PR1 | CTCGAGCGGCCGCCACTGTGCTGG | 9 |
| Region-PF1 | cctcagagtccgccacacccgaaagtAGCATCAA | 10 |
| | GAAGAACCTGATCGGAG | |
| Region-PR1 | ttgactagttgggatccagaacctccGCCCAGCA | 11 |
| | CCTTGAATTTCTTGC | |
| Region-PF2 | cctcagagtccgccacacccgaaagtGAGCGGCA | 12 |
| | CCCCATCTTCGG | |
| Region-PR2 | ttgactagttgggatccagaacctccCAGGAAGG | 13 |
| | ACTCTTCCAGTCTGTGG | |
| Region-PF3 | cctcagagtccgccacacccgaaagtGACCTGCG | 14 |
| | GCTGATCTATCTGGC | |
| Region-PR3 | ttgactagttgggatccagaacctccGGTGCTGT | 15 |
| | CCACCAGTTTCTTTCTCAG | |
| Region-PF4 | cctcagagtccgccacacccgaaagtGACCTGAA | 16 |
| | CCCCGACAACAGCG | |
| Region-PR4 | ttgactagttgggatccagaacctccGAAGTGGC | 17 |
| | CCCGGAACTTGATCA | |
| Region-PF5 | cctcagagtccgccacacccgaaagtGCCCCCCT | 18 |
| | GAGCGCCTCTATG | |
| Region-PR5 | ttgactagttgggatccagaacctccTCTCAGGA | 19 |
| | TGTCGCTCAGCAGGATG | |
| Region-PF6 | cctcagagtccgccacacccgaaagtCCCGTGGA | 20 |
| | AAACACCCAGCTG | |
| Region-PR6 | ttgactagttgggatccagaacctccCAGCTCTT | 21 |
| | TGATGCCCTCTTCGATC | |
| Region-PF7 | cctcagagtccgccacacccgaaagtTCCGAAGA | 22 |
| | GGTCGTGAAGAAGATGAAG | |
| Region-PR7 | ttgactagttgggatccagaacctccGGCCTTGT | 23 |
| | CGCTTCTGGTCAG | |
| Region-PF8 | cctcagagtccgccacacccgaaagtAGCGAACT | 24 |
| | GGATAAGGCCGGC | |
| Region-PR8 | ttgactagttgggatccagaacctccTCTCTCGG | 25 |
| | CCTTGGTCAGATTGTC | |
| Region-PF9 | cctcagagtccgccacacccgaaagtGTGAAAGT | 26 |
| | GATCACCCTGAAGTCCAAG | |
| Region-PR9 | ttgactagttgggatccagaacctccCTTAGTGT | 27 |
| | TCATCCGGGAGTCCAGG | |
| Region- | cctcagagtccgccacacccgaaagtGTGCGGAA | 28 |
| PF10 | GATGATCGCCAAGAG | |
| Region- | ttgactagttgggatccagaacctccCTTGTAGT | 29 |
| PR10 | CGCCGTACACGAACTCG | |

The expression vector plasmid pCDNA3.1 (+) (Miaoling Biology, P0157) was double-digested with Acc651 and EcoRI, and the linearized vector was recovered by agarose gel electrophoresis and gel extraction.

Using FokI-dCas9 (Miaoling Biology, P5109) as a template, PCR amplification was performed with the primer pair FokI-PF1 and FokI-PR1 in Table 2 to obtain the FokI coding sequence (SEQ ID NO: 5), which encodes the FokI domain with a linker.

Using pCDNA3.1-dCas9 (Miaoling Biology, P15213) as a template, PCR amplification was performed using different combinations of amplification primers for fragments encoding different fusion proteins in Table 3 (primer sequences are shown in Table 2), thereby obtaining different fragments.

### Proteins

| | | Amplified | Template |
|---|---|---|---|
| Plasmid Name | Primer 1 | Fragment | Plasmid |
| dCas9-Fokl-S1 | dCas9-PR1 | S1-F1 | pCDNA3.1-dCas9 |
| | dCas9-PF1 | S1-F2 | pCDNA3.1-dCas9 |
| dCas9-Fokl-S2 | dCas9-PR1 | S2-F1 | pCDNA3.1-dCas9 |
| | dCas9-PF1 | S2-F2 | pCDNA3.1-dCas9 |
| dCas9-Fokl-S3 | dCas9-PR1 | S3-F1 | pCDNA3.1-dCas9 |
| | dCas9-PF1 | S3-F2 | pCDNA3.1-dCas9 |
| dCas9-Fokl-S4 | dCas9-PR1 | S4-F1 | pCDNA3.1-dCas9 |
| | dCas9-PF1 | S4-F2 | pCDNA3.1-dCas9 |
| dCas9-Fokl-S5 | dCas9-PR1 | S5-F1 | pCDNA3.1-dCas9 |
| | dCas9-PF1 | S5-F2 | pCDNA3.1-dCas9 |
| dCas9-Fokl-S6 | dCas9-PR1 | S6-F1 | pCDNA3.1-dCas9 |
| | dCas9-PF1 | S6-F2 | pCDNA3.1-dCas9 |
| dCas9-Fokl-S7 | dCas9-PR1 | S7-F1 | pCDNA3.1-dCas9 |
| | dCas9-PF1 | S7-F2 | pCDNA3.1-dCas9 |
| dCas9-Fokl-S8 | dCas9-PR1 | S8-F1 | pCDNA3.1-dCas9 |
| | dCas9-PF1 | S8-F2 | pCDNA3.1-dCas9 |
| dCas9-Fokl-S9 | dCas9-PR1 | S9-F1 | pCDNA3.1-dCas9 |
| | dCas9-PF1 | S9-F2 | pCDNA3.1-dCas9 |
| dCas9-Fokl-S10 | dCas9-PR1 | S10-F1 | pCDNA3.1-dCas9 |
| | dCas9-PF1 | S10-F2 | pCDNA3.1-dCas9 |

The coding sequences of different fusion proteins, the FokI coding sequence, and the linearized vector were subjected to homologous recombination using Gibson Assembly® Master Mix (NEB), thereby integrating the fusion protein coding sequence into the cloning region of vector pCDNA3.1 (+) to construct recombinant vectors encoding the different structural fusion proteins listed in Table 1 (dCas9-FokI-S1 to dCas9-FokI-S10). For example, homologous recombination of linearized vector pCDNA3.1 (+)+fragment S1-F2+the FokI coding sequence+fragment S1-F1 yielded the dCas9-FokI-S1 vector.

The reaction mixture was transformed into Stbl3 competent cells, plated on LB plates with ampicillin resistance, cultured overnight at 37 C, and clones were picked for sequencing identification, thereby obtaining plasmid vectors encoding fusion proteins dCas9-FokI-S1 to dCas9-FokI-S10, respectively.

The amino acid sequence of the dCas9-FokI-S9 fusion protein is:

(SEQ ID NO: 3)
MKRPAATKKAGQAKKKKDKKYSIGLAIGTNSVGWAVITDEYKVPSKKFKV
LGNTDRHSIKKNLIGALLFDSGETAEATRLKRTARRRYTRRKNRICYLQE
IFSNEMAKVDDSFFHRLEESFLVEEDKKHERHPIFGNIVDEVAYHEKYPT
IYHLRKKLVDSTDKADLRLIYLALAHMIKFRGHFLIEGDLNPDNSDVDKL
FIQLVQTYNQLFEENPINASGVDAKAILSARLSKSRRLENLIAQLPGEKK
NGLFGNLIALSLGLTPNFKSNFDLAEDAKLQLSKDTYDDDLDNLLAQIGD
QYADLFLAAKNLSDAILLSDILRVNTEITKAPLSASMIKRYDEHHQDLTL
LKALVRQQLPEKYKEIFFDQSKNGYAGYIDGGASQEEFYKFIKPILEKMD
GTEELLVKLNREDLLRKQRTFDNGSIPHQIHLGELHAILRRQEDFYPFLK
DNREKIEKILTFRIPYYVGPLARGNSRFAWMTRKSEETITPWNFEEVVDK
GASAQSFIERMTNFDKNLPNEKVLPKHSLLYEYFTVYNELTKVKYVTEGM
RKPAFLSGEQKKAIVDLLFKTNRKVTVKQLKEDYFKKIECFDSVEISGVE
DRFNASLGTYHDLLKIIKDKDFLDNEENEDILEDIVLTLTLFEDREMIEE
RLKTYAHLFDDKVMKQLKRRRYTGWGRLSRKLINGIRDKQSGKTILDFLK
SDGFANRNFMQLIHDDSLTFKEDIQKAQVSGQGDSLHEHIANLAGSPAIK
KGILQTVKVVDELVKVMGRHKPENIVIEMARENQTTQKGQKNSRERMKRI
EEGIKELGSQILKEHPVENTQLQNEKLYLYYLQNGRDMYVDQELDINRLS
DYDVDHIVPQSFLKDDSIDNKVLTRSDKARGKSDNVPSEEVVKKMKNYWR
QLLNAKLITQRKFDNLTKAERGGLSELDKAGFIKRQLVETRQITKHVAQI
LDSRMNTKGGSGSQLVKSELEEKKSELRHKLKYVPHEYIELIEIARNSTQ
DRILEMKVMEFFMKVYGYRGKHLGGSRKPDGAIYTVGSPIDYGVIVDTKA
YSGGYNLPIGQADEMQRYVEENQTRNKHINPNEWWKVYPSSVTEFKFLFV
SGHFKGNYKAQLTRLNHITNCNGAVLSVEELLIGGEMIKAGTLTLEEVRR
KFNNGEINFSGSETPGTSESATPESVKVITLKSKLVSDFRKDFQFYKVRE
INNYHHAHDAYLNAVVGTALIKKYPKLESEFVYGDYKVYDVRKMIAKSEQ
EIGKATAKYFFYSNIMNFFKTEITLANGEIRKRPLIETNGETGEIVWDKG
RDFATVRKVLSMPQVNIVKKTEVQTGGFSKESILPKRNSDKLIARKKDWD
PKKYGGFDSPTVAYSVLVVAKVEKGKSKKLKSVKELLGITIMERSSFEKN
PIDFLEAKGYKEVKKDLIIKLPKYSLFELENGRKRMLASAGELQKGNELA
LPSKYVNFLYLASHYEKLKGSPEDNEQKQLFVEQHKHYLDEIIEQISEFS
KRVILADANLDKVLSAYNKHRDKPIREQAENIIHLFTLTNLGAPAAFKYF
DTTIDRKRYTSTKEVLDATLIHQSITGLYETRIDLSQLGGDSAGGGGSGG
GGSGGGGSGPKKKRKVAAAGS

The amino acid sequences of dCas9-FokI-S1 to dCas9-FokI-S8 and dCas9-FokI-S10 can be deduced by analogy, as they differ only in that the FokI domain with a linker is substituted at different positions shown in Table 1.

### Example 2: Construction of pCDH-CMV-EGFP-Reporter3-EF1a-Puro Cell Line

a. Construction of Lentiviral Expression Plasmid pCDH-CMV-EGFP-Reporter3-EF1a-Puro for the GFP Reporter System

An EGFP coding sequence comprising the detection system was synthesized. The synthetic fragment was digested with Xbal and Notl and ligated, using T4 DNA ligase (Thermo Scientific™, EL0012), into the vector pCDH-CMV-MCS-EF1-Puro (Youbao Biology, VT1480) digested with Xbal and Notl. The ligation product was transformed into Stbl3, plated on ampicillin-containing plates, cultured overnight at 37 C, and clones were picked for sequencing identification, thereby obtaining the pCDH-CMV-EGFP-Reporter3-EF1a-Puro vector (SEQ ID NO: 30).

The synthesized EGFP coding sequence is as follows:

(SEQ ID NO: 31)
Tctagagcgagaaaagccttgtttgccaccatggaacggctcggagatca
tcattgcgtcgcgaggtgagcaagggcgaggagctgttcaccggggtggt
gcccatcctggtcgagctggacggcgacgtaaacggccacaagttcagcg
tgtccggcgagggcgagggcgatgccacctacggcaagctgaccctgaag
ttcatctgcaccaccggcaagctgcccgtgccctggcccaccctcgtgac
caccctgacctacggcgtgcagtgcttcagccgctaccccgaccacatga
agcagcacgacttcttcaagtccgccatgcccgaaggctacgtccaggag
cgcaccatcttcttcaaggacgacggcaactacaagacccgcgccgaggt
gaagttcgagggcgacaccctggtgaaccgcatcgagctgaagggcatcg
acttcaaggaggacggcaacatcctggggcacaagctggagtacaactac
aacagccacaacgtctatatcatggccgacaagcagaagaacggcatcaa
ggtgaacttcaagatccgccacaacatcgaggacggcagcgtgcagctcg
ccgaccactaccagcagaacacccccatcggcgacggccccgtgctgctg
cccgacaaccactacctgagcacccagtccaagctgagcaaagaccccaa
cgagaagcgcgatcacatggtcctgctggagttcgtgaccgccgccggga
tcactctcggcatggacgagctgtacaagtaagcggccgc

The underlined portions are the target sites of the two gRNAs.

b. Lentiviral Packaging

The sequence-verified pCDH-CMV-EGFP-Reporter3-EF1a-Puro plasmid was mixed with the viral packaging helper plasmids pMD2.G (Miaoling Biology, P0262) and psPAX2 (Miaoling Biology, P0261) at a molar ratio of 1:1:1, and 293T cells were transfected with PEI. At 48 h after transfection, the culture supernatant was collected and filtered through a 0.45 μm membrane to obtain crude pCDH-CMV-EGFP-Reporter3-EF1a-Puro virus.

c. Construction of Detection Cell Line by Viral Infection of 293T Cells

293T cells were infected by adding crude pCDH-CMV-EGFP-Reporter3-EF1a-Puro virus at ¼ volume of the culture medium. After 48 h of infection, the medium was replaced, and puromycin at 2 μg/ml was added for selection. The selected cells were subjected to limiting dilution for monoclonal screening, and the resulting monoclonal cells constituted the detection cell line.

### Example 3: Test of Editing Efficiencies of dCas9-FokI Fusion Proteins Having Different Structures

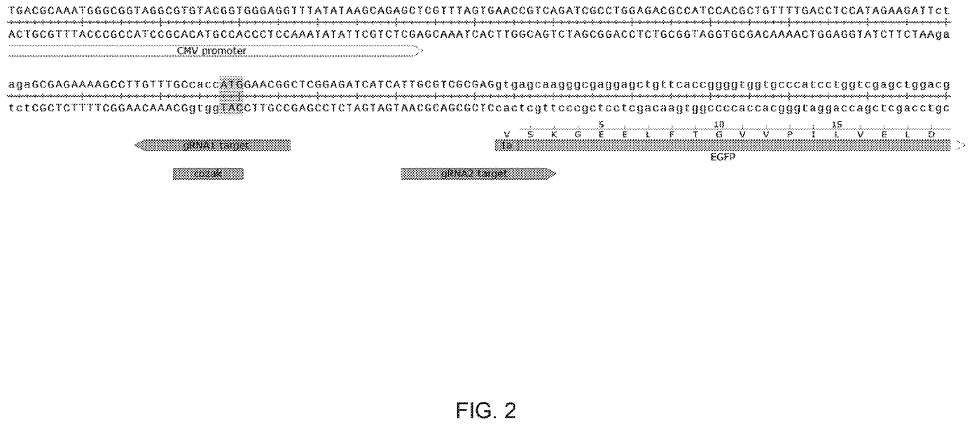

Because FokI can nick only a single strand of DNA, the inventors used dual gRNAs to guide fusion proteins of dCas9 and FokI to cleave double-stranded DNA. After cleavage, the DNA undergoes NHEJ repair in cells, thereby causing the GFP gene in some cells to regain a proper reading frame (as shown in FIG. 2), thus emitting fluorescence. The proportion of cells emitting green fluorescence, as detected by flow cytometry, was used to characterize the editing efficiencies of different dCas9-FokI fusion proteins.

Construction of gRNA Vectors

Guide-sequence coding sequences were introduced by restriction digestion-ligation, thereby obtaining two gRNA expression vectors, gRNA1-SpCas9-pUC57Kan (SEQ ID NO: 34) and gRNA2-SpCas9-PUC57Kan (SEQ ID NO: 35), in which expression of gRNA1 or gRNA2 can be driven by the U6 promoter. The gRNAs comprise guide sequences targeting EGFP and the corresponding SpCas9 gRNA scaffold sequence.

250 ng each of the recombinant plasmid vectors dCas9-FokI-S1 to dCas9-FokI-S9 from Example 1 were each mixed with 125 ng of gRNA1-SpCas9-pUC57Kan plasmid and 125 ng of gRNA2-SpCas9-pUC57Kan plasmid in 100 μl Opti-MEM (Gibco), followed by addition of 1.5 μl PEI (YEASEN), mixing well, and standing for 20 min. The mixture was then added to the detection cell line constructed in Example 2 for cell transfection. After 72 h of transfection, cells were collected for flow cytometric analysis.

The negative control (NC) group was the detection cell line of Example 2, to which no plasmid related to the CRISPR system was transfected.

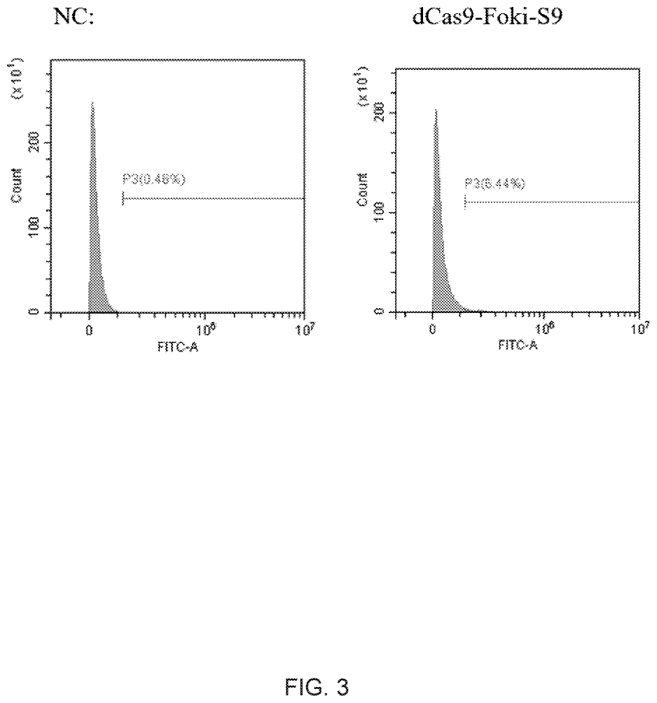

The results are shown in FIG. 3 and Table 4.

TABLE 4
Editing Efficiency

| Group | Proportion of Fluorescent Cells |
|---|---|
| NC | 0.46% |
| dCas9-Fokl-S1 | 0.82% |
| dCas9-Fokl-S2 | 1.05% |
| dCas9-Fokl-S3 | 1.24% |
| dCas9-Fokl-S4 | 1.20% |
| dCas9-Fokl-S5 | 1.05% |
| dCas9-Fokl-S6 | 1.31% |
| dCas9-Fokl-S7 | 1.32% |
| dCas9-Fokl-S8 | 2.55% |
| dCas9-Fokl-S9 | 6.44% |
| dCas9-Fokl-S10 | 1.45% |

Flow cytometry results showed that the editing efficiency of dCas9-FokI-S9 was much higher than that of dCas9-FokI-S1 to dCas9-FokI-S8 and dCas9-FokI-S10.

### Example 4: Construction of dCas9-FokI-S9 Control Plasmids and PAMless Plasmid

Using a method similar to that in Example 1, dCas9-FokI-953-01 plasmid (SEQ ID NO: 36) and dCas9-FOKI-953-02 plasmid (SEQ ID NO: 37) were constructed, both for expression of fusion proteins in which FokI is internally fused within dSpCas9. The difference between the two is that the recombinant protein expressed by dCas9-FOKI-953-01 plasmid contains the FokI domain inserted directly between amino acids 953 and 954; whereas the recombinant protein expressed by dCas9-FOKI-953-02 plasmid contains the FokI domain inserted between amino acids 953 and 954 with a terminal linker sequence for connection to the Cas9 sequence.

Using a method similar to that in Example 1, dCas9PAMless-FokI-S9 plasmid (SEQ ID NO: 38) was constructed for expression of a recombinant protein in which the FokI domain is internally inserted into dead PAMless Cas9 (SpRY variant).

### Example 5: Editing of Reporter System by dCas9-FokI Fusion Proteins Having Different Structures

Different fusion proteins were obtained by fusing Cas9 with FokI. According to the experimental scheme described in Example 3, editing effects were preliminarily verified in the reporter system. After different fusion proteins were combined with gRNAs, the EGFP gene was edited and, upon generation of indels, expression of fluorescent EGFP was restored. The editing effects of the fusion proteins were determined by detecting the proportion of eGFP-positive cells.

The dCas9-FokI positive control group was transfected with 250 ng of dCas9-FokI plasmid (Miaoling Biology P5109, expressing dCas9 with N-terminally fused FokI), and 125 ng each of gRNA1 plasmid and gRNA2 plasmid.

The Cas9 nickase positive control group was transfected with 250 ng of Cas9 nickase plasmid (SEQ ID NO: 39, expressing Cas9 nickase), and 125 ng each of gRNA1 plasmid and gRNA2 plasmid.

The dCas9-FokI-S9 group, dCas9PAMless-FokI-S9 experimental group, dCas9-FokI-953-01 group, and dCas9-FokI-953-02 group were transfected with 250 ng of the corresponding recombinant protein plasmid and 125 ng each of gRNA1 plasmid and gRNA2 plasmid.

The SpCas9-gRNA1+2 control group was transfected with 250 ng of pX459 V2.0 plasmid (expressing wild-type SpCas9), and 125 ng each of gRNA1 plasmid and gRNA2 plasmid.

The SpCas9-gRNA1 control group was transfected with 250 ng of pX459 V2.0 plasmid (expressing wild-type SpCas9) and 250 ng of gRNA1 plasmid.

The SpCas9-gRNA2 control group was transfected with 250 ng of pX459 V2.0 plasmid (expressing wild-type SpCas9) and 250 ng of gRNA2 plasmid.

The negative control (NC) group was the detection cell line of Example 2, to which no plasmid related to the CRISPR system was transfected.

Three transfection reagents, namely PEI, EndoFectin, and Lipo2000, were tested separately.

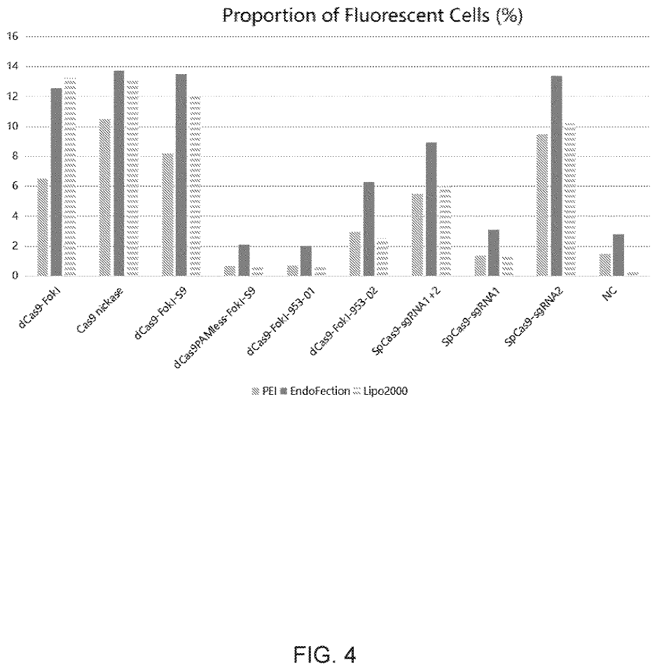

The specific results are shown in Table 5 and FIG. 4. It can be seen that the editing efficiency of the dCas9-FokI-S9 fusion protein in this reporter system was comparable to that of Cas9 nickase, and even superior to that of SpCas9. It was also much higher than that of fusion proteins in which FokI was inserted or substituted at position 953 aa, and superior to the fusion protein in which the FokI domain was terminally fused to dCas9 portion.

TABLE 5
Editing Efficiencies of Different Recombinant Proteins

Proportion of Fluorescent Cells (%)

| Group | PEI | EndoFection | Lipo2000 |
|---|---|---|---|
| dCas9-Fokl | 6.49 | 12.56 | 13.23 |
| Cas9 nickase | 10.5 | 13.73 | 13.2 |
| dCas9-Fokl-S9 | 8.19 | 13.49 | 12.15 |
| dCas9PAMless-Fokl- | 0.65 | 2.10 | 0.68 |
| S9 | | | |
| dCas9-Fokl-953-01 | 0.72 | 1.99 | 0.72 |
| dCas9-Fokl-953-02 | 2.95 | 6.30 | 2.51 |
| SpCas9-gRNA1 + 2 | 5.52 | 8.94 | 6.02 |
| SpCas9-gRNA1 | 1.38 | 3.08 | 1.30 |
| SpCas9-gRNA2 | 9.50 | 13.38 | 10.22 |
| NC | 1.47 | 2.80 | 0.29 |

### Example 6: Test of Endogenous Gene Editing Efficiency of dCas9-FokI-S9

Plasmids expressing gRNAs targeting the HBB gene and the VEGFA gene, respectively, each comprising the corresponding SpCas9 gRNA scaffold sequence (i.e., SEQ ID NO: 40), were constructed, with expression driven by the U6 promoter.

The guide sequences of the gRNAs are shown in Table 6.

TABLE 6
Detection of Endogenous Gene
Editing Efficiency

| gRNA | Guide Sequence | SEQ ID NO |
|---|---|---|
| HBB-gRNA01 | CTTGCCCCACAGGGCAGTAA | 45 |
| HBB-gRNA02 | TTGGTGGTGAGGCCCTGGGC | 46 |
| VEGFA-gRNA01 | GGGTGGGGGGAGTTTGCTCC | 47 |
| VEGFA-gRNA02 | ATTCCCTCTTTAGCCAGAGC | 48 |
| VEGFA-gRNA03 | TCCCTCTTTAGCCAGAGCCG | 49 |

250 ng of the recombinant protein plasmid from the foregoing examples and 250 ng of the gRNA plasmid from this example (250 ng when one gRNA plasmid was used, or about 125 ng each when two gRNA plasmids were used) were mixed, followed by addition of 1.5 μl PEI, and the mixture was transfected into 293T cells.

After 72 h of transfection, the cells were washed once with PBS, digested with trypsin, and collected by centrifugation at 300 rcf for 5 min, followed by resuspension in 500 μl PBS. 50 μl of the resuspended cells were taken, 48 μl DirectPCR Lysis Reagent (Cell) and 2 μl proteinase K were added for treatment, and 1 μl of the treated lysate was used for PCR amplification. The primer sequences for amplification are shown in Table 7.

TABLE 7
Amplification Primers for Target
Sequences HBB and VEGFA

| Primer | | SEQ ID |
|---|---|---|
| Name | Primer Sequence | NO |
| HBB-PF1 | CAAGGGTAGACCACCAGCAGCCTA | 41 |
| HBB-PR1 | GCCAGTGCCAGAAGAGCCAAGG | 42 |
| VEGFA- | GGAACCACACAGCTTCCCGTTCTC | 43 |
| PF1 | | |
| VEGFA- | CCTGAGAGCCGTTCCCTCTTTGC | 44 |
| PR1 | | |

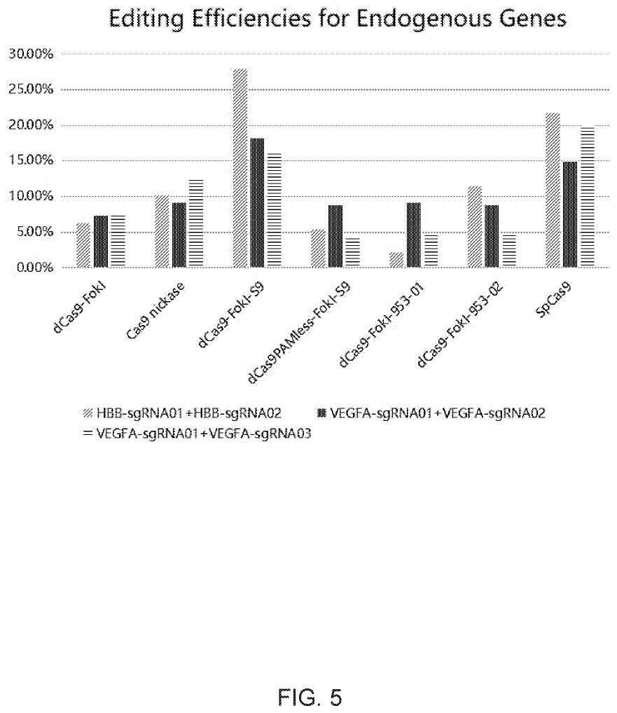

The amplified products were subjected to Sanger sequencing, and the sequencing results were analyzed by TIDE to determine editing efficiency. The results are shown in Table 8 and FIG. 5.

TABLE 8
Editing Efficiencies for Endogenous Genes

| | HBB- | VEGFA- | VEGFA- |
|---|---|---|---|
| | gRNA01 + | gRNA01 + | gRNA01 + |
| | HBB- | VEGFA- | VEGFA- |
| Recombinant Protein | gRNA02 | gRNA02 | gRNA03 |
| dCas9-Fokl | 6.3% | 7.3% | 7.4% |
| Cas9 nickase | 10.2% | 9.1% | 12.3% |
| dCas9-Fokl-S9 | 27.9% | 18.2% | 16% |
| dCas9PAMless-Fokl-S9 | 5.4% | 8.8% | 4.4% |
| dCas9-Fokl-953-01 | 2.2% | 9.1% | 4.7% |
| dCas9-Fokl-953-02 | 11.4% | 8.8% | 4.7% |
| SpCas9 | 21.70% | 14.9% | 20% |

TIDE analysis showed that dCas9-FokI-S9 had good editing effects on endogenous genes, with editing efficiency comparable to that of SpCas9 (slightly better in some cases), and markedly superior to terminally fused FokI dCas9-FokI, dCas9-FokI-953-01 and dCas9-FokI-953-02 in which FokI was inserted or substituted at position 953 aa, and PAMless Cas9, and also superior to Cas9 nickase.

Although specific embodiments of the present disclosure have been described above, those skilled in the art will appreciate that these are merely illustrative. Various changes or modifications may be made to these embodiments without departing from the principles and spirit of the present disclosure. Accordingly, the scope of protection of the present disclosure is defined by the appended claims.

What is claimed is:

1. A fusion protein comprising:
(a) a dCas9 portion; and
(b) a functional domain,
wherein the dCas9 portion is a remaining part of a dCas9 protein in which one or more consecutive amino acid residues located in a random coil region of the dCas9 protein are replaced by the functional domain,
wherein the random coil region is located in the RuvC domain of the dCas9 protein, and
wherein the dCas9 protein is a Cas9 protein lacking nuclease activity.

2. The fusion protein of claim 1, wherein the fusion protein specifically binds to a target nucleic acid under the guidance of a guide polynucleotide, and the guide polynucleotide includes a guide sequence.

3. The fusion protein of claim 1, wherein the functional domain is selected from the group consisting of a deaminase domain, a methylation domain, a demethylation domain, a subcellular localization signal, a transcriptional activation domain, a transcriptional repression domain, and a nuclease domain.

4. The fusion protein of claim 1, wherein the functional domain is a nuclease domain.

5. The fusion protein of claim 1, wherein the nuclease domain is a FokI domain.

6. The fusion protein of claim 1, wherein, under the guidance of a guide polynucleotide, the fusion protein binds to a double-stranded target nucleic acid and nicks at least one strand of the target nucleic acid.

7. The fusion protein of claim 1, wherein the fusion protein forms a complex with a guide polynucleotide, and the guide polynucleotide comprises a guide sequence engineered to direct the complex to specifically bind to a double-stranded target nucleic acid.

8. The fusion protein of claim 7, wherein the guide polynucleotide further comprises a scaffold sequence, and the scaffold sequence interacts with the dCas9 portion of the fusion protein.

9. The fusion protein of claim 1, wherein the dCas9 protein comprises an amino acid sequence having at least 80% sequence identity to SEQ ID NO: 1.

10. The fusion protein of claim 1, wherein the dCas9 protein is derived from SpCas9, SaCas9, NmeCas9, StCas9, or CjCas9.

11. The fusion protein of claim 1, wherein the functional domain is linked to the dCas9 portion via one or more linkers.

12. The fusion protein of claim 1, wherein the functional domain replaces 1 to 20 consecutive amino acid residues of the dCas9 protein.

13. The fusion protein of claim 1, wherein the one or more consecutive amino acid residues replaced by the functional domain are located within at least one of amino acid positions 37-41, 107-113, 147-149, 169-172, 308-314, 792-799, 864-871, 906-908, 943-952, and 1015-1017 of SEQ ID NO: 2.

14. A gene editing system comprising:
(a) the fusion protein of claim 1, or a nucleic acid encoding the fusion protein; and
(b) a guide polynucleotide, or a nucleic acid encoding the guide polynucleotide.

15. The gene editing system of claim 14, wherein the dCas9 protein comprises an amino acid sequence having at least 80% sequence identity to SEQ ID NO: 1.

16. The gene editing system of claim 14, wherein the functional domain is selected from the group consisting of a deaminase domain, a methylation domain, a demethylation domain, a subcellular localization signal, a transcriptional activation domain, a transcriptional repression domain, and a nuclease domain.

17. The gene editing system of claim 14, wherein the functional domain replaces 1 to 20 consecutive amino acid residues of the dCas9 protein.

18. A polynucleotide encoding the fusion protein of claim 1.

19. A pharmaceutical composition comprising:
(a) the fusion protein of claim 1;
(b) a gene editing system comprising the fusion protein of claim 1, or a nucleic acid encoding the fusion protein and; and a guide polynucleotide, or a nucleic acid encoding the guide polynucleotide; or
(c) a polynucleotide encoding the fusion protein of claim 1,
and optionally a pharmaceutically acceptable carrier and/or excipient.

20. A method of modifying a target nucleic acid or modulating expression of a target gene, the method comprising contacting the target nucleic acid, or a cell comprising the target nucleic acid, with a component selected from the group consisting of:
(a) the fusion protein of claim 1;
(b) a gene editing system comprising the fusion protein of claim 1, or a nucleic acid encoding the fusion protein; and a guide polynucleotide, or a nucleic acid encoding the guide polynucleotide;
(c) a polynucleotide encoding the fusion protein of claim 1; or
(d) a pharmaceutical composition comprising any one of (a) to (c) and optionally a pharmaceutically acceptable carrier and/or excipient,
thereby modifying the target nucleic acid and/or modulating expression of the target gene.


SEQUENCE LISTING (52 sequences)

SEQ ID NO: 1 - protein, 1368 aa, Streptococcus pyogenes [sequence omitted]

SEQ ID NO: 2 - protein, 1368 aa, synthetic construct
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKARGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 3 - protein, 1621 aa, synthetic construct
MKRPAATKKAGQAKKKKDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKARGKSDNVPSEEVVKKMKNYWRQLLNAKLITQRKFDNLTKAERGGLSELDKAGFIKRQLVETRQITKHVAQILDSRMNTKGGSGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFSGSETPGTSESATPESVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSAGGGGSGGGGSGGGGSGPKKKRKVAAAGS

SEQ ID NO: 4 - protein, 217 aa, synthetic construct
GGSGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFSGSETPGTSESATPES

SEQ ID NO: 5 - DNA, 651 bp, synthetic construct
  source 1..651: mol_type = other DNA
ggaggttctggatcccaactagtcaaaagtgaactggaggagaagaaatctgaacttcgtcataaattgaaatatgtgcctcatgaatatattgaattaattgaaattgccagaaattccactcaggatagaattcttgaaatgaaggtaatggaattttttatgaaagtttatggatatagaggtaaacatttgggtggatcaaggaaaccggacggagcaatttatactgtcggatctcctattgattacggtgtgatcgtggatactaaagcttatagcggaggttataatctgccaattggccaagcagatgaaatgcaacgatatgtcgaagaaaatcaaacacgaaacaaacatatcaaccctaatgaatggtggaaagtctatccatcttctgtaacggaatttaagtttttatttgtgagtggtcactttaaaggaaactacaaagctcagcttacacgattaaatcatatcactaattgtaatggagctgttcttagtgtagaagagcttttaattggtggagaaatgattaaagccggcacattaaccttagaggaagtcagacggaaatttaataacggcgagataaactttagcggcagcgagactcccgggacctcagagtccgccacacccgaaagt

SEQ ID NO: 6 - DNA, 29 bp, synthetic construct
  source 1..29: mol_type = other DNA
ggaggttctggatcccaactagtcaaaag

SEQ ID NO: 7 - DNA, 25 bp, synthetic construct
  source 1..25: mol_type = other DNA
actttcgggtgtggcggactctgag

SEQ ID NO: 8 - DNA, 27 bp, synthetic construct
  source 1..27: mol_type = other DNA
caagctggctagcgtttaaacttaagc

SEQ ID NO: 9 - DNA, 24 bp, synthetic construct
  source 1..24: mol_type = other DNA
ctcgagcggccgccactgtgctgg

SEQ ID NO: 10 - DNA, 51 bp, synthetic construct
  source 1..51: mol_type = other DNA
cctcagagtccgccacacccgaaagtagcatcaagaagaacctgatcggag

SEQ ID NO: 11 - DNA, 49 bp, synthetic construct
  source 1..49: mol_type = other DNA
ttgactagttgggatccagaacctccgcccagcaccttgaatttcttgc

SEQ ID NO: 12 - DNA, 46 bp, synthetic construct
  source 1..46: mol_type = other DNA
cctcagagtccgccacacccgaaagtgagcggcaccccatcttcgg

SEQ ID NO: 13 - DNA, 51 bp, synthetic construct
  source 1..51: mol_type = other DNA
ttgactagttgggatccagaacctcccaggaaggactcttccagtctgtgg

SEQ ID NO: 14 - DNA, 49 bp, synthetic construct
  source 1..49: mol_type = other DNA
cctcagagtccgccacacccgaaagtgacctgcggctgatctatctggc

SEQ ID NO: 15 - DNA, 53 bp, synthetic construct
  source 1..53: mol_type = other DNA
ttgactagttgggatccagaacctccggtgctgtccaccagtttctttctcag

SEQ ID NO: 16 - DNA, 48 bp, synthetic construct
  source 1..48: mol_type = other DNA
cctcagagtccgccacacccgaaagtgacctgaaccccgacaacagcg

SEQ ID NO: 17 - DNA, 49 bp, synthetic construct
  source 1..49: mol_type = other DNA
ttgactagttgggatccagaacctccgaagtggccccggaacttgatca

SEQ ID NO: 18 - DNA, 47 bp, synthetic construct
  source 1..47: mol_type = other DNA
cctcagagtccgccacacccgaaagtgcccccctgagcgcctctatg

SEQ ID NO: 19 - DNA, 51 bp, synthetic construct
  source 1..51: mol_type = other DNA
ttgactagttgggatccagaacctcctctcaggatgtcgctcagcaggatg

SEQ ID NO: 20 - DNA, 47 bp, synthetic construct
  source 1..47: mol_type = other DNA
cctcagagtccgccacacccgaaagtcccgtggaaaacacccagctg

SEQ ID NO: 21 - DNA, 51 bp, synthetic construct
  source 1..51: mol_type = other DNA
ttgactagttgggatccagaacctcccagctctttgatgccctcttcgatc

SEQ ID NO: 22 - DNA, 53 bp, synthetic construct
  source 1..53: mol_type = other DNA
cctcagagtccgccacacccgaaagttccgaagaggtcgtgaagaagatgaag

SEQ ID NO: 23 - DNA, 47 bp, synthetic construct
  source 1..47: mol_type = other DNA
ttgactagttgggatccagaacctccggccttgtcgcttctggtcag

SEQ ID NO: 24 - DNA, 47 bp, synthetic construct
  source 1..47: mol_type = other DNA
cctcagagtccgccacacccgaaagtagcgaactggataaggccggc

SEQ ID NO: 25 - DNA, 50 bp, synthetic construct
  source 1..50: mol_type = other DNA
ttgactagttgggatccagaacctcctctctcggccttggtcagattgtc

SEQ ID NO: 26 - DNA, 53 bp, synthetic construct
  source 1..53: mol_type = other DNA
cctcagagtccgccacacccgaaagtgtgaaagtgatcaccctgaagtccaag

SEQ ID NO: 27 - DNA, 51 bp, synthetic construct
  source 1..51: mol_type = other DNA
ttgactagttgggatccagaacctcccttagtgttcatccgggagtccagg

SEQ ID NO: 28 - DNA, 49 bp, synthetic construct
  source 1..49: mol_type = other DNA
cctcagagtccgccacacccgaaagtgtgcggaagatgatcgccaagag

SEQ ID NO: 29 - DNA, 51 bp, synthetic construct
  source 1..51: mol_type = other DNA
ttgactagttgggatccagaacctcccttgtagtcgccgtacacgaactcg

SEQ ID NO: 30 - DNA, 8131 bp, synthetic construct
  source 1..8131: mol_type = other DNA
acgcgtgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacaataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccactgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagattggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaattcaaaattttatcgatactagtattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtttatataagcagagctcgtttagtgaaccgtcagatcgcctggagacgccatccacgctgttttgacctccatagaagattctagagcgagaaaagccttgtttgccaccatggaacggctcggagatcatcattgcgtcgcgaggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccaagctgagcaaagaccccaacgagaagcgcgatcacatggtcctgctggagttcgtgaccgccgccgggatcactctcggcatggacgagctgtacaagtaagcggccgcgaaggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgctagatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggtacctttaagaccaatgacttacaaggcagctgtagatcttagccactttttaaaagaaaaggggggactggaagggctaattcactcccaacgaaaataagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccagttccgcccattctccgccccatggctgactaattttttttatttatgcagaggccgaggccgcctcggcctctgagctattccagaagtagtgaggaggcttttttggaggcctagacttttgcagagacggcccaaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctg

SEQ ID NO: 31 - DNA, 790 bp, synthetic construct
  source 1..790: mol_type = other DNA
tctagagcgagaaaagccttgtttgccaccatggaacggctcggagatcatcattgcgtcgcgaggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccaagctgagcaaagaccccaacgagaagcgcgatcacatggtcctgctggagttcgtgaccgccgccgggatcactctcggcatggacgagctgtacaagtaagcggccgc

SEQ ID NO: 32 - DNA, 240 bp, synthetic construct
  source 1..240: mol_type = other DNA
tgacgcaaatgggcggtaggcgtgtacggtgggaggtttatataagcagagctcgtttagtgaaccgtcagatcgcctggagacgccatccacgctgttttgacctccatagaagattctagagcgagaaaagccttgtttgccaccatggaacggctcggagatcatcattgcgtcgcgaggtgagcaagggcgaggagctgttcaccggggtggtgcccatcctggtcgagctggacg

SEQ ID NO: 33 - protein, 19 aa, synthetic construct
VSKGEELFTGVVPILVELD

SEQ ID NO: 34 - DNA, 2749 bp, synthetic construct
  source 1..2749: mol_type = other DNA
tcgcgcgtttcggtgatgacggtgaaaacctctgacacatgcagctcccggagactgtcacagcttgtctgtaagcggatgccgggagcagacaagcccgtcagggcgcgtcagcgggtgttggcgggtgtcggggctggcttaactatgcggcatcagagcagattgtactgagagtgcaccatagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgccgttccatggtggcaaacagttttagagctagaaatagcaagttaaaataaggctagtccgttatcaacttgaaaaagtggcaccgagtcggtgcttttttatcccaatggcgcgccgagcttggctcgagcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctgttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgaaacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggcccttttgtc

SEQ ID NO: 35 - DNA, 2749 bp, synthetic construct
  source 1..2749: mol_type = other DNA
tcgcgcgtttcggtgatgacggtgaaaacctctgacacatgcagctcccggagactgtcacagcttgtctgtaagcggatgccgggagcagacaagcccgtcagggcgcgtcagcgggtgttggcgggtgtcggggctggcttaactatgcggcatcagagcagattgtactgagagtgcaccatagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgttgcgtcgcgaggtgagcaagttttagagctagaaatagcaagttaaaataaggctagtccgttatcaacttgaaaaagtggcaccgagtcggtgcttttttatcccaatggcgcgccgagcttggctcgagcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctgttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgaaacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggcccttttgtc

SEQ ID NO: 36 - DNA, 10237 bp, synthetic construct
  source 1..10237: mol_type = other DNA
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgccaccatgaaaaggccggcggccacgaaaaaggccggccaggcaaaaaagaaaaaggacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccacatcgtgcctcagagctttctgaaggacgactccatcgacaacaaggtgctgaccagaagcgacaaggcccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgcaactagtcaaaagtgaactggaggagaagaaatctgaacttcgtcataaattgaaatatgtgcctcatgaatatattgaattaattgaaattgccagaaattccactcaggatagaattcttgaaatgaaggtaatggaattttttatgaaagtttatggatatagaggtaaacatttgggtggatcaaggaaaccggacggagcaatttatactgtcggatctcctattgattacggtgtgatcgtggatactaaagcttatagcggaggttataatctgccaattggccaagcagatgaaatgcaacgatatgtcgaagaaaatcaaacacgaaacaaacatatcaaccctaatgaatggtggaaagtctatccatcttctgtaacggaatttaagtttttatttgtgagtggtcactttaaaggaaactacaaagctcagcttacacgattaaatcatatcactaattgtaatggagctgttcttagtgtagaagagcttttaattggtggagaaatgattaaagccggcacattaaccttagaggaagtcagacggaaatttaataacggcgagataaactttaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacagcgctggaggaggtggaagcggaggaggaggaagcggaggaggaggtagcggacctaagaaaaagaggaaggtggcggccgctggatcctagaattctgcagatatccagcacagtggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 37 - DNA, 10267 bp, synthetic construct
  source 1..10267: mol_type = other DNA
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgccaccatgaaaaggccggcggccacgaaaaaggccggccaggcaaaaaagaaaaaggacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccacatcgtgcctcagagctttctgaaggacgactccatcgacaacaaggtgctgaccagaagcgacaaggcccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtggcatctgcttctcaactagtcaaaagtgaactggaggagaagaaatctgaacttcgtcataaattgaaatatgtgcctcatgaatatattgaattaattgaaattgccagaaattccactcaggatagaattcttgaaatgaaggtaatggaattttttatgaaagtttatggatatagaggtaaacatttgggtggatcaaggaaaccggacggagcaatttatactgtcggatctcctattgattacggtgtgatcgtggatactaaagcttatagcggaggttataatctgccaattggccaagcagatgaaatgcaacgatatgtcgaagaaaatcaaacacgaaacaaacatatcaaccctaatgaatggtggaaagtctatccatcttctgtaacggaatttaagtttttatttgtgagtggtcactttaaaggaaactacaaagctcagcttacacgattaaatcatatcactaattgtaatggagctgttcttagtgtagaagagcttttaattggtggagaaatgattaaagccggcacattaaccttagaggaagtcagacggaaatttaataacggcgagataaacttttctcctgcgtcagaggttaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacagcgctggaggaggtggaagcggaggaggaggaagcggaggaggaggtagcggacctaagaaaaagaggaaggtggcggccgctggatcctagaattctgcagatatccagcacagtggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 38 - DNA, 10270 bp, synthetic construct
  source 1..10270: mol_type = other DNA
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgccaccatgaaaaggccggcggccacgaaaaaggccggccaggcaaaaaagaaaaaggacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgagagaacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagggaggttctggatcccaactagtcaaaagtgaactggaggagaagaaatctgaacttcgtcataaattgaaatatgtgcctcatgaatatattgaattaattgaaattgccagaaattccactcaggatagaattcttgaaatgaaggtaatggaattttttatgaaagtttatggatatagaggtaaacatttgggtggatcaaggaaaccggacggagcaatttatactgtcggatctcctattgattacggtgtgatcgtggatactaaagcttatagcggaggttataatctgccaattggccaagcagatgaaatgcaacgatatgtcgaagaaaatcaaacacgaaacaaacatatcaaccctaatgaatggtggaaagtctatccatcttctgtaacggaatttaagtttttatttgtgagtggtcactttaaaggaaactacaaagctcagcttacacgattaaatcatatcactaattgtaatggagctgttcttagtgtagaagagcttttaattggtggagaaatgattaaagccggcacattaaccttagaggaagtcagacggaaatttaataacggcgagataaactttagcggcagcgagactcccgggacctcagagtccgccacacccgaaagtgtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcagacccaagaggaacagcgataagctgatcgccagaaagaaggactgggaccctaagaagtacggcggcttcctgtggcccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccaagcagctgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccagactgggagcccctagagccttcaagtactttgacaccaccatcgaccccaagcagtacagaagcaccaaagaggtgctggacgccaccctgatccaccagagcatcaccggcctgtacgagacacggatcgacctgtctcagctgggaggtgacagcgctggaggaggtggaagcggaggaggaggaagcggaggaggaggtagcggacctaagaaaaagaggaaggtggcggccgctggatcctagaattctgcagatatccagcacagtggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 39 - DNA, 9553 bp, synthetic construct
  source 1..9553: mol_type = other DNA
cagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctctagctagaggtcgacggtatacagacatgataagatacattgatgagtttggacaaaccacaactagaatgcagtgaaaaaaatgctttatttgtgaaatttgtgatgctattgctttatttgtaaccattataagctgcaataaacaagttggggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatccgaaaatggatatacaagctcccgggagctttttgcaaaagcctaggcctccaaaaaagcctcctcactacttctggaatagctcagaggcagaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagaattaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtggggataccccctagagccccagctgcgcagatctgctatggcagggcctgccgccccgacgttggctgcgagccctgggccttcacccgaacttggggggtggggtggggaaaaggaagaaacgcgggcgtattggccccaatggggtctcggtggggtatcgacagagtgccagccctgggaccgaaccccgcgtttatgaacaaacgacccaacacccgtgcgttttattctgtctttttattgccgtcatagcgcgggttccttccggtattgtctccttccgtgtttcagttagcctcccccgtttaaactcattactaaccggtagggatcgaaccctttcacaccttcctcttcttcttggggtcagccctgctgtctccaccgagctgagagaggtcgattcttgtttcatagagccccgtaattgactgatgaatcagtgtggcgtccaggacctcctttgtagaggtgtaccgctttctgtctatggtggtgtcgaagtacttgaaggctgcaggcgcgcccaagttggtcagagtaaacaagtggataatgttttctgcctgctccctgatgggcttatccctgtgcttattgtaagcagaaagcaccttatcgaggttagcgtcggcgaggatcactcttttggagaattcgcttatttgctcgatgatctcatcaaggtagtgtttgtgttgttccacgaacagctgcttctgctcattatcttcgggagaccctttgagcttttcatagtggctggccagatacaagaaattaacgtatttagagggcagtgccagctcgttacctttctgcagctcgcccgcactagcgagcattcgtttccggccgttttcaagctcaaagagagagtacttgggaagcttaatgatgaggtcttttttgacctctttatatcctttcgcctcgagaaagtcgatggggtttttttcgaagcttgatcgctccatgattgtgatgcccagcagttccttgacgcttttgagttttttagacttccctttctccactttggccacaaccagtacactgtaagcgactgtaggagaatcgaatccgccgtatttcttggggtcccaatcttttttgcgtgcgatcagcttgtcgctgttccttttcgggaggatactttccttggagaagcctccggtctgtacttcggtctttttaacgatgttcacctgcggcatggacaggaccttccggactgtcgcgaaatccctacccttgtcccacacgatttctcctgtttctccgtttgtttcgataagtggtcgcttccgaatctctccattggccagtgtaatctcggtcttgaaaaaattcataatattgctgtaaaagaagtacttagcggtggccttgcctatttcctgctcagactttgcgatcattttcctaacatcgtacactttatagtctccgtaaacaaattcagattcaagcttgggatattttttgataagtgcagtgcctaccactgcattcaggtaggcatcatgcgcatggtggtaattgttgatctctctcaccttataaaactgaaagtcctttctgaaatctgagaccagcttagacttcagagtaataactttcacctctcgaatcagtttgtcattttcatcgtacttggtgttcatgcgtgaatcgagaatttgggccacgtgcttggtgatctggcgtgtctcaacaagctgccttttgatgaagccggctttatccaactcagacaggccacctcgttcagccttagtcagattatcgaacttccgttgtgtgatcagtttggcgttcagcagctgccgccaataatttttcattttcttgacaacttcttctgaggggacgttatcactcttccctctatttttatcggatcttgtcaacactttattatcaatagaatcatctttgagaaaagactggggcacgatatgatccacgtcgtagtcggagagccgattgatgtccagttcctgatccacgtacatgtccctgccgttctgcaggtagtacaggtagagcttctcattctgaagctgggtgttttcaactgggtgttccttaaggatttgggaccccagttcttttataccctcttcaatcctcttcatcctttccctactgttcttctgtcccttctgggtagtttggttctctcgggccatctcgataacgatattctcgggcttatgccttcccattactttgacgagttcatccacgaccttaacggtctgcagtattccctttttgatagctgggctacctgcaagattagcgatgtgctcgtgaagactgtccccctggccagaaacttgtgctttctggatgtcctccttaaaggtgagagagtcatcatggatcaactgcatgaagttcctgttggcaaatccatcggacttaagaaaatccaggattgtctttccactctgcttgtctcggatcccattgatcagttttcttgacagccgcccccatcctgtatatcggcgcctcttgagctgtttcatgactttgtcgtcgaagagatgagcgtaagttttcaagcgttcttcaatcatctccctatcttcaaacaacgtaagggtgaggacaatgtcctcaagaatgtcctcgttctcctcattgtccaggaagtccttgtctttaatgattttcaggagatcgtgatacgttcccagggatgcgttgaagcgatcctccactccgctgatttcaacagagtcgaaacattcaatctttttgaaatagtcttctttgagctgtttcacggtaactttccggttcgtcttgaagaggaggtccacgatagctttcttctgctctccagacaggaatgctggctttctcatcccttctgtgacgtatttgaccttggtgagctcgttataaactgtgaagtactcgtacagcagagagtgtttaggaagcaccttttcgttaggcagatttttatcaaagttagtcatcctttcgatgaaggactgggcagaggcccccttatccacgacttcctcgaagttccagggagtgatggtctcttctgatttgcgagtcatccacgcgaatctggaatttccccgggcgagggggcctacatagtagggtatccgaaatgtgaggattttctcaatcttttccctgttatctttcaaaaaggggtagaaatcctcttgccgcctgaggatagcgtgcagttcgcccaggtgaatctggtgggggatgcttccattgtcgaaagtgcgctgtttgcgcaacagatcttctctgttaagctttaccagcagctcctcggtgccgtccattttttccaagatgggcttaataaatttgtaaaattcctcctggcttgctccgccgtcaatgtatccggcgtagccatttttagactgatcgaagaaaatttccttgtacttctcaggcagttgctgtctgacaagggccttcagcaaagtcaagtcttggtggtgctcatcatagcgcttgatcatactagcgctcagcggagctttggtgatctccgtgttcactcgcagaatatcactcagcagaatggcgtctgacaggttctttgccgccaaaaaaaggtctgcgtactggtcgccgatctgggccagcagattgtcgagatcatcatcgtaggtgtctttgctcagttgaagcttggcatcttcggccaggtcgaagttagatttaaagttgggggtcagcccgagtgacagggcgataagattaccaaacaggccgttcttcttctccccagggagctgtgcgatgaggttttcgagccgccgggatttggacagcctagcgctcaggattgctttggcgtcaactccggatgcgttgatcgggttctcttcgaaaagctgattgtaagtctgaaccagttggataaagagtttgtcgacatcgctgttgtctgggttcaggtccccctcgatgaggaagtgtccccgaaatttgatcatatgcgccagcgcgagatagatcaaccgcaagtcagccttatcagtactgtctacaagcttcttcctcagatgatatatggttgggtacttttcatggtacgccacctcgtccacgatattgccaaagattgggtggcgctcgtgctttttatcctcctccaccaaaaaggactcctccagcctatggaagaaagagtcatccaccttagccatctcattactaaagatctcctgcaggtagcagatccgattctttctgcgggtatatctgcgccgtgctgttcttttgagccgcgtggcttcggccgtctccccggagtcgaacaggagggcgccaatgaggttcttctttatgctgtggcgatcggtattgcccagaactttgaattttttgctcggcaccttgtactcgtccgtaatgacggcccagccgacgctgtttgtgccgatagcgagcccaatggagtacttcttgtccatggtggcaagggttcgatcctctagagtccggaggctggatcggtcccggtgtcttctatggaggtcaaaacagcgtggatggcgtctccaggcgatctgacggttcactaaacgagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactagtcaataatcaatgtcaacgcgtatatctggcccgtacatcgcgaagcagcgcaaaacgcctaaccctaagcagattcttcatgcaattgtcggtcaagccttgccttgttgtagcttaaattttgctcgcgcactactcagcgacctccaacacacaagcagggagcagatactggcttaactatgcggcatcagagcagattgtactgagagtgcaccataggggatcgggagatctcccgatccgtcgacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatg

SEQ ID NO: 40 - DNA, 76 bp, synthetic construct
  source 1..76: mol_type = other DNA
gttttagagctagaaatagcaagttaaaataaggctagtccgttatcaacttgaaaaagtggcaccgagtcggtgc

SEQ ID NO: 41 - DNA, 24 bp, synthetic construct
  source 1..24: mol_type = other DNA
caagggtagaccaccagcagccta

SEQ ID NO: 42 - DNA, 22 bp, synthetic construct
  source 1..22: mol_type = other DNA
gccagtgccagaagagccaagg

SEQ ID NO: 43 - DNA, 24 bp, synthetic construct
  source 1..24: mol_type = other DNA
ggaaccacacagcttcccgttctc

SEQ ID NO: 44 - DNA, 23 bp, synthetic construct
  source 1..23: mol_type = other DNA
cctgagagccgttccctctttgc

SEQ ID NO: 45 - DNA, 20 bp, synthetic construct
  source 1..20: mol_type = other DNA
cttgccccacagggcagtaa

SEQ ID NO: 46 - DNA, 20 bp, synthetic construct
  source 1..20: mol_type = other DNA
ttggtggtgaggccctgggc

SEQ ID NO: 47 - DNA, 20 bp, synthetic construct
  source 1..20: mol_type = other DNA
gggtggggggagtttgctcc

SEQ ID NO: 48 - DNA, 20 bp, synthetic construct
  source 1..20: mol_type = other DNA
attccctctttagccagagc

SEQ ID NO: 49 - DNA, 20 bp, synthetic construct
  source 1..20: mol_type = other DNA
tccctctttagccagagccg

SEQ ID NO: 50 - protein, 5 aa, synthetic construct
GGSGS

SEQ ID NO: 51 - protein, 16 aa, synthetic construct
SGSETPGTSESATPES

SEQ ID NO: 52 - protein, 196 aa, synthetic construct
QLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINF